SEQUENCE LISTING (43 sequences)

SEQ ID NO: 1 - DNA, 24 bp, homo sapiens
agccatgaccacctttctacctca

SEQ ID NO: 2 - DNA, 20 bp, Homo sapiens
tcatacccaagaaagcacct

SEQ ID NO: 3 - DNA, 20 bp, Artificial sequence
  gRNA targeting miR-Let7c binding site
ctgaggtagaaaggtgatca

SEQ ID NO: 4 - DNA, 20 bp, Artificial sequence
  gRNA targeting miR-Let7c binding site
ctgaggtagaaaggtggtca

SEQ ID NO: 5 - DNA, 20 bp, Artificial sequence
  gRNA targeting miR-Let7c binding site
atggatctgaggtagaaagg

SEQ ID NO: 6 - DNA, 20 bp, Artificial sequence
  gRNA targeting miR-Let7c-binding site
aagatggatctgaggtagaa

SEQ ID NO: 7 - DNA, 20 bp, Artificial sequence
  gRNA targeting miR-196-b binding site
gtgctttcttgggtatgaca

SEQ ID NO: 8 - DNA, 20 bp, Artificial sequence
  gRNA targeting miR-196-b binding site
ctttaaataggtgctttctt

SEQ ID NO: 9 - DNA, 20 bp, Artificial sequence
  gRNA targeting ERF binding site
tcttccggaacaaagttgct

SEQ ID NO: 10 - DNA, 20 bp, Artificial sequence
  gRNA targeting ERF binding site
gaacaaagttgctgggccgg

SEQ ID NO: 11 - DNA, 20 bp, Artificial sequence
  gRNA targeting ERF binding site
acgtagtggggctgatcttc

SEQ ID NO: 12 - DNA, 20 bp, Artificial sequence
  gRNA targeting ERF binding site
ccggcccagcaactttgttc

SEQ ID NO: 13 - DNA, 20 bp, Artificial sequence
  gRNA targeting ERF binding site
atcttccggaacaaagttgc

SEQ ID NO: 14 - DNA, 20 bp, Artificial sequence
  gRNA targeting ERF binding site
tcttccggaacaaagttgct

SEQ ID NO: 15 - DNA, 20 bp, Artificial sequence
  gRNA targeting EN1 binding site 2
atcagccccactacgttccc

SEQ ID NO: 16 - DNA, 20 bp, Artificial sequence
  gRNA targeting EN1 binding site 2
gctgacccgggaacgtagtg

SEQ ID NO: 17 - DNA, 20 bp, Artificial sequence
  gRNA targeting EN1 binding site 2
acgctgacccgggaacgtag

SEQ ID NO: 18 - DNA, 52 bp, homo sapiens
ttatataaaaaggaaagccatgaccacctttctacctcagatccatcttcat

SEQ ID NO: 19 - DNA, 50 bp, homo sapiens
ttttatcaggccatgtcatacccaagaaagcacctatttaaagaaaaaac

SEQ ID NO: 20 - DNA, 36 bp, Homo sapiens
agtggggctgatcttccggaacaaagttgctgggcc

SEQ ID NO: 21 - DNA, 37 bp, Homo sapiens
cgctgacccgggaacgtagtggggctgatcttccgga

SEQ ID NO: 22 - DNA, 26 bp, Artificial sequence
  Forward primer 5' of Utrophin A
acgaattcagtgacatcattaagtcc

SEQ ID NO: 23 - DNA, 26 bp, Artificial sequence
  Reverse primer 5' of utrophin A
atccatttggtaaaggttttcttctg

SEQ ID NO: 24 - DNA, 20 bp, artificial sequence
  gRNA targeting miR-150-133b-296-5p(II) binding site
ttattttagaataggttggg

SEQ ID NO: 25 - DNA, 20 bp, artificial sequence
  gRNA targeting miR-Let7c-binding site
aaggtggttctgaggtagaa

SEQ ID NO: 26 - DNA, 54 bp, artificial sequence
  synthetic polynucleotide
ttatataaaaaggaaagccatgaccacctttctacctcagatccatcttcatcc

SEQ ID NO: 27 - RNA, 20 nt, artificial sequence
  synthetic polynucleotide (gRNA)
cugagguagaaaggugauca

SEQ ID NO: 28 - RNA, 20 nt, artificial sequence
  synthetic polynucleotide (gRNA)
cugagguagaaaggugguca

SEQ ID NO: 29 - RNA, 20 nt, artificial sequence
  synthetic polynucleotide (gRNA)
auggaucugagguagaaagg

SEQ ID NO: 30 - RNA, 20 nt, artificial sequence
  synthetic polynucleotide (gRNA)
aagauggaucugagguagaa

SEQ ID NO: 31 - RNA, 20 nt, artificial sequence
  synthetic polynucleotide (gRNA)
aaggugguucugagguagaa

SEQ ID NO: 32 - RNA, 20 nt, artificial sequence
  synthetic polynucleotide (gRNA)
gugcuuucuuggguaugaca

SEQ ID NO: 33 - RNA, 20 nt, artificial sequence
  synthetic polynucleotide (gRNA)
cuuuaaauaggugcuuucuu

SEQ ID NO: 34 - RNA, 20 nt, artificial sequence
  synthetic polynucleotide (gRNA)
ucuuccggaacaaaguugcu

SEQ ID NO: 35 - RNA, 20 nt, artificial sequence
  synthetic polynucleotide (gRNA)
gaacaaaguugcugggccgg

SEQ ID NO: 36 - RNA, 20 nt, artificial sequence
  synthetic polynucleotide (gRNA)
acguaguggggcugaucuuc

SEQ ID NO: 37 - RNA, 20 nt, artificial sequence
  synthetic polynucleotide (gRNA)
ccggcccagcaacuuuguuc

SEQ ID NO: 38 - RNA, 20 nt, artificial sequence
  synthetic polynucleotide (gRNA)
aucuuccggaacaaaguugc

SEQ ID NO: 39 - RNA, 20 nt, artificial sequence
  synthetic polynucleotide (gRNA)
ucuuccggaacaaaguugcu

SEQ ID NO: 40 - RNA, 20 nt, artificial sequence
  synthetic polynucleotide (gRNA)
aucagccccacuacguuccc

SEQ ID NO: 41 - RNA, 20 nt, artificial sequence
  synthetic polynucleotide (gRNA)
gcugacccgggaacguagug

SEQ ID NO: 42 - RNA, 20 nt, artificial sequence
  synthetic polynucleotide (gRNA)
acgcugacccgggaacguag

SEQ ID NO: 43 - DNA, 2046 bp, Homo sapiens
gcaatgtgaagtattcatccggccaaccaatgtttcctgacgtacagtgttgcccttttcagcaaatgccaattccaagttccattaaatcagaagctccatggctccttggcccacgatgttgagtgctgactgtgtgttctactgaaagagtaaaacactgactatccaaagagaaatggatattttgtttttataataaccatatattattgttttcttcttccctttctatgcaagtgtaaattaatgaacagagaggtatttggaaatggtaatacatttgtcacggatttgtataatgtatacagcattgggaaagtgggtgggggctttctaatatgataccgtctttttaataactatgacaaagcttacataagaattagaagaccactttacatttttacattccttctgctgttcatattaaccttgcacaattacttcattttttctttgactcttttaccacaatgttttggttatttataatttatcagccatatgtttatcagccatataaccaactagatcccaaatagatccatgtatttgtttccgtgatttggccacattaataaattcataaatttcaatcaaatatcttatatatacacacatatggtttaagctacagccctgtgtatgccgtttaactttatttgacgttgcccacttacttctttgctgaccacttggataaccgtaataaaaatcctataagcctaaatggcatttcttttgggatatttttcctgcattttattccctttttatataagtaggaattaattatttattttatgtcttaatctatttgataaagaagactacattataataatctcaaagatcatattaccaaaggttgcccacttgagcatattttcattttgacacagaaacaaaatttagtacaacctttcctagttcccatgtcttgattttcatcattacatgcacagcagacctttacctattgtgataccagaacacatcattgtctttggttcccttcaaagagaattttattgttgttttgtattttcaagtccttaatagttcttgaaactcctagttgttttcttgttgaaagcagacacacatttagtgcacggcttattttacctttcgggtgaaagatcagatgtttttatacccttcacttgatcaatatatttggaaagaatgtttatcaaaagtctatgtcactgcttctacagaagaatgaaattaatgcttaggtgatggtacctccacctacatctttttgagtgcattcaattatgtattttggtttagcttctgatttaacatttaattgattcagtttaaacatgttacttaattagcaaatgtagaggaaccaaaaaaaggtgaaaataatatgttttgattcaaacctaaagacataaaaacataaagacattttaactttgggttctctttagctgggatctggccagaaggaggcttaaagttagaaattgctattattttagaataggttgggtgggttggggggcaagggtgtctatttgcagcagagatattttgaaaagaagaaaattgttttatataaaaaggaaagccatgaccacctttctacctcagatccatcttcatccattgcattggaaactgctttatgctgctgcagtctgcaaagtctagagcttttatcaggccatgtcatacccaagaaagcacctatttaaagaaaaaacaattccctgagctctcaactccaagttgtagatttggtgtcttccttgttcttactttaaaaagtcatgtgttaattttttttctgcctgtatttgtatgcaaaatgtcctctatctgctattaaagaaaagctacgtaaaacactacattgtaaccttctaagtaataataaataaaaagaaatatattgcagtaacaatgggaagtaagtatgtagttcttttgaaatatgtggtaaagaactaatcacagactatcatctaatctggttacatattgtatttttcatcctgaataaaagtaattttaacacaa

(19) United States
(12) Patent Application Publication
(10) Pub. No.: US 2023/0272433 A1
(43) Pub. Date: Aug. 31, 2023

(54) Enhancing Utrophin Expression in Cell by Inducing Mutations Within Utrophin Regulatory Elements and Therapeutic Use Thereof

(71) Applicants: Genethon, Evry-Courcouronnes (FR); Universite d'Evry Val d'Essonne, Evry (FR); INSERM( INSTITUT NATIONAL DE LA SANTÉ ET DE LA RECHERCHE MÉDICALE), Paris (FR)

(72) Inventors: Mario Amendola, Paris (FR); Simon Guiraud, Saint Pierre du Perray (FR)

(73) Assignees: Genethon, Evry-Courcouronnes (FR); Universite d'Evry Val d'Essonne, Evry (FR); Institut National de la Santé et de la Recherche Médicale, Paris (FR)

(21) Appl. No.: 18/024,102

(22) PCT Filed: Sep. 29, 2021
(86) PCT No.: PCT/EP2021/076882

§ 371 Date: Mar. 1, 2023

(30) Foreign Application Priority Data
Sep. 29, 2020 (EP) 20306112.2

(51) Int. Cl. C12N 15/90; C12N 15/11; C12N 9/22; C12N 5/077

(52) U.S. Cl. CPC C12N 15/907; C12N 5/0658; C12N 9/22; C12N 15/11; C12N 2310/20; C12N 2800/80

(57) ABSTRACT

The invention relates to a composition for enhancing utrophin expression in cell by inducing mutations within a target sequence comprising a utrophin repressor binding site using a gene editing enzyme and the use thereof for the treatment of a dy-strophinopathy.

### FIELD OF THE INVENTION

The invention relates to a composition for enhancing utrophin expression in cell by inducing mutations within a target sequence comprising a utrophin regulatory element using a gene editing enzyme. The invention relates also to the therapeutic use thereof for the treatment of a dystrophinopathy.

### BACKGROUND

Duchenne muscular dystrophy (DMD) is a lethal X-linked neuromuscular disorder caused by mutations in the dystrophin gene. The disease affects 1 in 5000 new-born males and is one of the most common recessive disorders in the human population. In the absence of the dystrophin protein, the link between the cytoskeleton and extracellular matrix is impaired resulting in loss of muscle strength, flexibility and stability. DMD patients are restricted to wheelchair by the age of 12 years and usually succumb in their second to fourth decade of life due to cardio-respiratory failure. Although considerable progress has been made in gene-based, cell-based and pharmacological strategies, there is currently no effective treatment for DMD.

Among gene-based strategies, exon-skipping and stop codon read-through show limited efficacy and are only applicable to a specific subset of DMD patients. The use of exon-skipping commonly refers to the use of synthetic antisense oligonucleotide to inhibit a splice enhancer site to prevent a particular exon from participating in splicing (Mann CJ, et al. Proc Natl Acad Sci U S A. 2001 Jan 2; 98(1):42-7). Exon-skipping approach can only be used in certain patients that may have a deletion where the reading frame could be restored by skipping an additional exon adjacent to the deletion (Perry B Shieh, Neurotherapeutics. 2018 Oct;15(4):840-848). Gene therapy using recombinant associated adenovirus (rAAV) and micro-dystrophin is currently the most promising approach (see Sakamoto M. et al., Biochem Biophys Res Commun. 2002 May 17; 293 (4): 1265-72) but safety and efficacy of the treatment remains to be assessed. Furthermore, this approach delivers a truncated and partially functional dystrophin and not recapitulates the benefit of the full-length dystrophin. An alternative therapeutic approach, applicable to all DMD patients, and potentially also to Becker patients, irrespective of their genetic defect, consists in upregulating utrophin, a structural and functional paralogue of dystrophin able to compensate for the primary defect in DMD. Previous studies demonstrated that a 3-fold increase of utrophin levels rescue the dystrophic pathophysiology in different animal models of DMD without any toxic effect. Several mechanisms were previously described to upregulate utrophin expression at the gene, mRNA and protein levels. For instance, in W02015/018503, a recombinant adeno-associated virus (AAV) vector comprising a gene encoding a fusion protein comprising a transcriptional activation element fused to a Zinc finger protein allowing utrophin expression increase is disclosed. In the same manner, a dual-AAV system was developed using a combination of Cas9 with deactivated nuclease activity (dCas9) fused to a transcription activation domain. Co-injection of AAV-dCas9 with an AAV-gRNA targeting utrophin allows to improve the muscular dystrophy symptoms in mdx mice model of DMD (see Liao H. et al., Cell. 2017 Dec 14; 171(7): 1495- 507). A similar approach is disclosed in WO 2020/101042.

Another approach was to target utrophin repressor elements to upregulate utrophin expression. Indeed, in the 5′UTR/promoter-enhancer region, a number of utrophin trans-repressors have been identified (e.g. EN1, EN2 and Ets-2). Utrophin is also subject to repression by several miRNAs (e.g. Let7c, miR-206) and cis-AU-Rich repressor sequences on the 3′UTR region. Recently, a 2′-O-methy oligonucleotides blocking the let7 miR binding site on Utrophin mRNA was shown to upregulate utrophin protein expression by 2 and 3-fold, respectively in murine myoblasts cells and in the mdx murine model of DMD (WO 2019/183005). The upregulation of utrophin was associated with significant histological and functional improvements (Mishra et al., PLoS One. 2017, 12(10):e0182676). Inhibition of miRNA using anti-sense sequences in C2C12 cells has also been described in WO2009/134710. However, these approaches need a continuous expression of the transgene to activate utrophin expression and it remains a need to develop strategy wherein expecting modification inducing utrophin upregulation is permanent.

Recently, CRISPR/Cas9-based approach was used in vitro in immortalized human myoblasts for deleting 3′UTR region of utrophin (UTRN) gene comprising miRNA binding sites (Soblechero-Martin et al., 2020, bioR_{X}iv preprint; doi.org/10.1101/2020.02.24.962316; Kasturi Sengupta et al., Molecular therapy: Nucleic Acids, 2020, 22). The deletion of the complete region of regulatory element may induce mRNA instability and misexpression of utrophin provoking eventual side effects.

### SUMMARY

The inventors have developed a novel utrophin upregulation-based therapeutic strategy for DMD using gene editing enzyme such as CRISPR-Cas system to disrupt repressor domains on the utrophin promoter or to perturb the binding site of miRs or other RNA destabilizing element in order to respectively de-repress utrophin transcription and translation and therefore upregulate utrophin levels. Contrary to previous methods such as exon-skipping/oligonucleotide, this strategy acts at the DNA level and the expected modification will be therefore permanent. In contrast to deletion of the complete region of a regulatory element, the inventors used here gene editing enzyme such as CRISPR/Cas with a single guide RNA, to induce mutations precisely within the target sequence. In contrast to the deletion of the regulatory elements, the method used herein allows to maintain the stability of the regulatory elements adjacent to targeted repressor binding site(s) and reduce side effects. Moreover, in comparison to traditional gene therapy based on truncated protein lacking important binding sites, a specific targeting strategy to upregulate the endogenous utrophin will generate the full length utrophin, with a better therapeutic and immunological potential. Using this strategy, the inventors particularly showed that the specific disruption of Let7c binding site, miR-196b binding site, ERF binding site and EN1 binding site 2 allows to increase efficiently utrophin expression in comparison to other repressor binding sites. Surprisingly, the specific disruption of a single repressor binding site, in particular Let7c binding site, increased utrophin expression as efficiently as the deletion of the complete region of the repressor binding site comprising a cluster of repressor binding sites. In mdx mice model of DMD, co-administration of rAAV expressing Cas9 and rAAV expressing single gRNA targeting Let7c binding site improved muscle architecture and histology compared to control. This opens new perspectives for the treatment of dystrophinopaties.

The present invention relates to a method for enhancing utrophin expression in a cell, comprising introducing into a cell a composition comprising at least one gene editing enzyme capable of inducing sequence-specific mutation(s) within a target sequence comprising a repressor binding site of utrophin gene selected from the group consisting of Ets-2-repressor factor (ERF) binding sites, preferably consisting of sequence CGGAA, homeobox protein engrailed-1 (EN1) binding site 2, preferably consisting of GTAGTGG, Let7c binding site, preferably consisting of SEQ ID NO: 1 and miR-196b binding site, preferably consisting of SEQ ID NO: 2, and wherein the mutation(s) disrupt the repressor binding site without deleting the whole repressor binding site sequence.

In a particular embodiment, said gene editing enzyme is a site-specific nuclease, a base editor or prime editor, more particularly a CRISPR/Cas gene editing enzyme comprising a guide RNA that comprises a complementary sequence to said target sequence comprising a utrophin repression binding site. In a preferred embodiment, said gRNA comprises a sequence selected from the group consisting of SEQ ID NO: 3-17 and 25.

In a particular embodiment, when said composition comprises at least two gene editing enzymes which are sequence-specific nucleases said nucleases are used successively in such a way that a first sequence-specific nuclease induces a first site-specific mutation event within a target sequence and once first mutation event is repaired, a second sequence-specific nuclease is used to induce a second site-specific mutation event within a target sequence.

In another aspect, the present invention relates to a composition for enhancing utrophin expression comprising at least one gene editing enzyme capable of inducing site-specific mutations within a target sequence comprising at least one repressor binding site of utrophin gene selected from the group consisting of: Ets-2-repressor factor (ERF) binding sites, preferably consisting of sequence CGGAA, homeobox protein engrailed-1 (EN1) binding site 2, preferably consisting of sequence GTAGTGG, Let7c binding site, preferably consisting of SEQ ID NO: 1 and miR-196b binding site, preferably consisting of SEQ ID NO: 2 and wherein the mutation(s) disrupt the repressor binding site without deleting the whole repressor binding site sequence. In some preferred embodiments, the repressor binding site of utrophin gene is Let7c binding site, preferably consisting of SEQ ID NO: 1.

In particular embodiment, said composition comprises a CRISPR/Cas gene editing enzyme comprising a guide RNA that comprises a complementary sequence to said target sequence comprising said utrophin repression binding site. In a preferred embodiment, said gRNA comprises a sequence selected from the group consisting of SEQ ID NO: 3-17 and 25.

In particular embodiment, said gene editing enzyme is encoded by a nucleic acid construct, preferably including in a viral vector, more preferably an AAV vector.

In a preferred embodiment, the present invention relates to the composition as described above for use in the treatment of dystrophinopathy, preferably Duchenne Muscular Dystrophy, Becker Muscular Dystrophy or X-linked dilated cardiomyopathy.

In another aspect, the present invention relates to a pharmaceutical composition comprising a composition as described above and a pharmaceutical excipient and its use in the treatment of dystrophinopathy, preferably Duchenne Muscular Dystrophy, Becker Muscular Dystrophy or X-linked dilated cardiomyopathy.

In another aspect, the present invention relates to an engineered cell comprising site-specific mutations within at least one target sequence comprising a repressor binding site of utrophin gene selected from the group consisting of Let7c binding site, miR-196b binding site, ERF binding site or and EN1 binding site 2; preferably Let7c binding site; and wherein the mutations disrupt the repressor binding site without deleting the whole repressor binding site sequence and its use in the treatment of dystrophinopathy, more preferably Duchenne Muscular Dystrophy, Becker Muscular Dystrophy or X-linked dilated cardiomyopathy.

### BRIEF DESCRIPTION OF THE FIGURES

FIG. 1: Schematic organisation of the utrophin gene with associated promoters and key repressor domains. Utrophin A promoter is CpG rich at the 5′-end and contains an E-Box and N-box motifs controlling the synaptic expression. Ets-2 repressor factor silences extrasynaptic utrophin expression through the N-Box and EN1 is also able to inhibit utrophin expression. On the 3′UTR of utrophin, several miRs post-transcriptionally repress utrophin expression. Exons (grey boxes with exon numbers) and intronic regions (black line), intronic enhancer (DUE for utrophin), untranslated first exon 1A of utrophin are specified. Arrows indicate transcription start sites.

FIG. 2: Enhancement of Utrophin A expression after treatment with Cas9-RNP-sgRNA targeting utrophin repressor binding sites. Human DMD myoblasts were exposed to Cas9-RNP and sgRNA targeting the binding site of Let7c, miR-196b, miR150/133b/296-5p(II) or ERB (ERF binding site) for 48 hours (three biological replicates). Utrophin transcripts were normalised with gapdh. Values are mean ± SEM of n = 3 per condition; *P < 0.05, **P < 0.01, ***P < 0.001.

FIG. 3: Reporter system and 3′UTR variants of utrophin. Several 3′UTR constructs with specific deleted area were synthetized and inserted downstream of the Gaussia luciferase gene in the dual reporter plasmid pEZX-GA02. Nucleotide positions are denoted on the right.

FIG. 4: Reporter gene expression according to specific 3′UTR variants. All pEZX-GA02-3′UTR constructs were transfected in hDMD D52 myoblasts. After 48 hours, the Gaussia luciferease level was measured and normalized by the SEAP expression. n = 3/group; ∗P < 0.05, ∗∗P < 0.01, ∗∗∗P < 0.01 relative to C1 - Full Length. Data are presented as mean ± SEM.

FIG. 5: Utrophin protein expression after sgRNA-Cas9 treatment. Relative utrophin protein expression in human DMD D52 myoblasts treated with SpCas9 and hEN1, hERB or hLet7c1 guide was determined by western blot and standardized for α-actinin loading. Relative utrophin expression is shown as mean ± SEM of n = 2 per condition.

FIG. 6: Utrophin mRNA expression after sgRNA-Cas9 treatment in hDMD D52 myoblasts. Relative utrophin A mRNA level in hDMD D52 myoblasts after 5 days of treatment with SpCas9-gRNA. Utrophin transcripts were normalised with gapdh. Values are mean ± SEM of n = 3 per condition; *P < 0.05, **P < 0.01, ***P < 0.001. Percentage of Indels are indicated.

FIG. 7: Utrophin mRNA expression after sgRNA-Cas9 treatment in C2C12 myoblasts. Relative utrophin A mRNA level in C2C12 myoblasts after 5 days of treatment with SpCas9-gRNA. Utrophin transcripts were normalised with gapdh. Values are mean ± SEM of n = 2 per condition;

FIG. 8: Utrophin protein expression after sgRNA-Cas9 treatment. Relative utrophin protein expression in healthy murine C2Cl2 myoblasts treated with SpCas9 and mLet7c2 and hLet7c2 guides was determined by western blot and standardized for tubulin loading.. Relative utrophin expression is shown as mean ± SEM of n = 2 per condition.

FIG. 9: Utrophin mRNA expression after sgRNA-Cas9 treatment in hDMD D52 myoblasts. Relative utrophin A mRNA level after 5 days of treatment with SpCas9-hLet7c2 or mLet7c2. Different cas9:gRNA ratio were used as well as different enhancer concentration. Initial condition: cas9:gRNA ratio of 1:2; enhancer concentration 1×. Optimized condition: cas9:gRNA ratio of 1:5; enhancer concentration 5×. Utrophin transcripts were normalised with gapdh. Values are mean ± SEM of n = 3 per condition; *P < 0.05, **P < 0.01, ***P < 0.001.

FIG. 10. In vivo assessment of the rAAV-mLet7c2/rAAV-SpCas9 treatment in mdx mice. (A) Relative utrophin protein expression in mdx TA tissue treated with rAAV-Rosa26/rAAV-SpCas9 (Control) or with rAAV-mLet7c2/rAAV-SpCas9 was determined by western blot and standardized for α-actinin loading. Relative utrophin expression is shown as mean ± SEM of n = 3 per condition. (B) Immunofluoresence staining for utrophin in TA muscles of 9 weeks old mdx mice treated for 5 weeks with 1^{E}12vg total dose of rAAV-mLet7c2/rAAV-SpCas9 or rAAV-Rosa26/rAAV-SpCas9 (control). Transverse sections were stained with anti-utrophin monoclonal antibody SC-33700 and anti-mouse secondary antibody. Magnification: 20×. (C) Hematoxylin-eosin-stained transverse muscle sections of TA muscle (9 weeks of age) in control vs rAAV-mLet7c2/rAAV-SpCas9 treated mdx mice showing necrotic areas (black stars) and regenerating fibres (black arrows). Magnification: 20×. (D) Quantification of centronucleation and necrosis/inflammation in treated rAAV-Rosa26/rAAV-SpCas9 (control) and rAAV-mLet7c2/rAAV-SpCas9 transverse muscle sections (C). Values are mean ± SEM of n = 3 per groups; *P < 0.05.

### DETAILED DESCRIPTION

### Composition for Enhancing Utrophin Expression in Cell

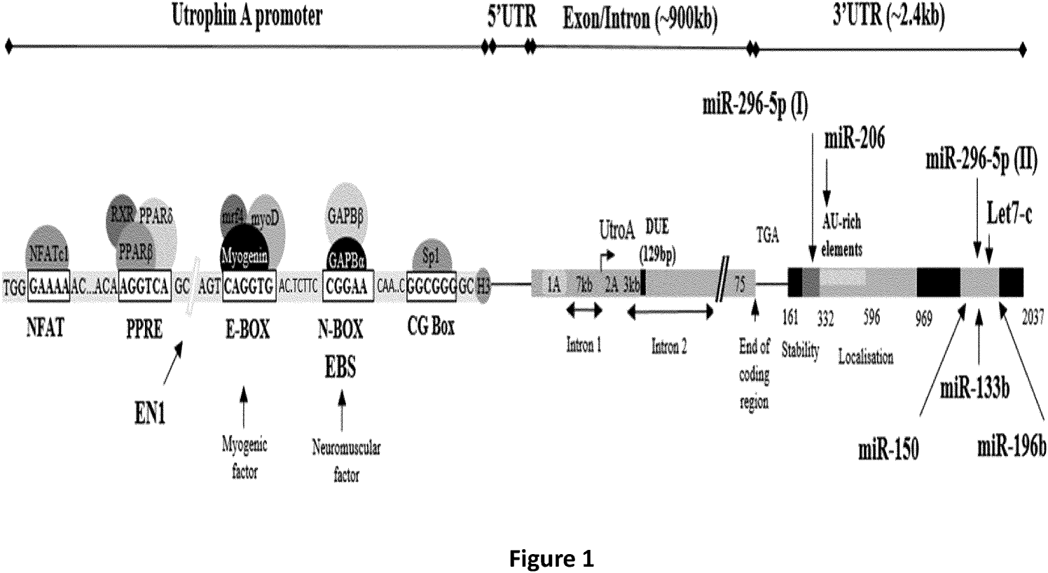

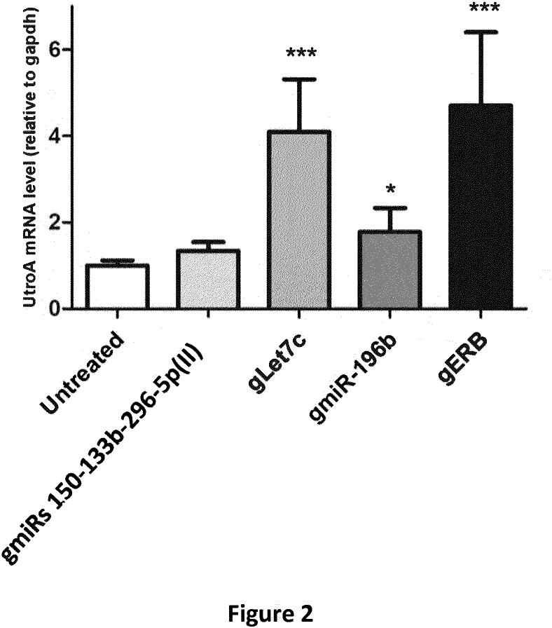

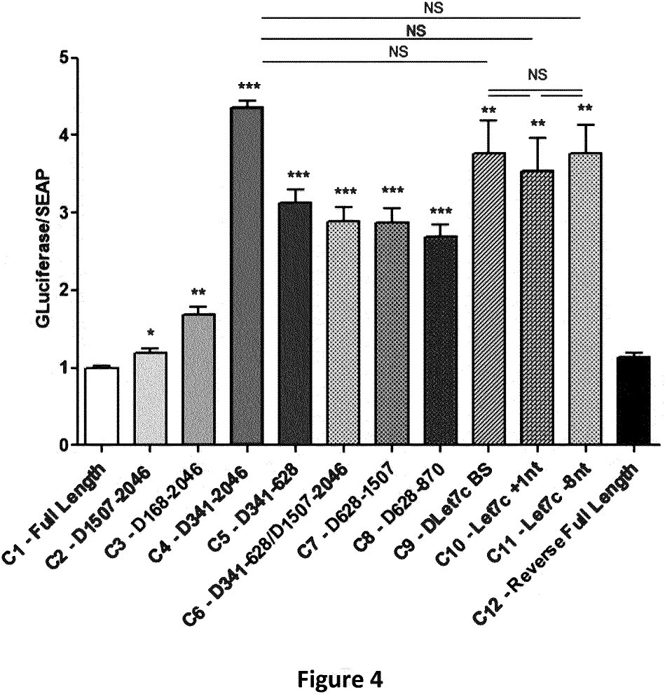

Utrophin expression is controlled by several regulatory elements adjacent to the utrophin gene (FIG. 1). For instance, the AU-rich element in the 3′-UTR modulates mRNA stability. The deletion of a regulatory element may induce mRNA instability and induces misexpression of utrophin gene. In contrast to deletion of the complete region of a regulatory element, the inventors used here gene editing enzyme to induce site-specific mutations precisely within target sequence and in contrast to the deletion of the regulatory element, the method used herein allows to maintain the stability of the regulatory element(s) adjacent to targeted repressor region(s) and reduces side effects. Moreover, for a clinical aspect, the present method inducing site-specific mutations within target sequence comprising utrophin repressor binding site results in: 1) easier delivery; 2) higher efficiency of expected modifications; 3) lower risks of off-targets, chromosomal translocation and aberrations; 4) less toxic genomic double strand breaks per cells, compared to the prior art method inducing deletion of the regulatory element. Using this strategy, the inventors particularly showed that the specific disruption of Let7c binding site, miR-196b binding site, ERF binding site and EN1 binding site 2 allows to increase efficiently utrophin expression in comparison to other repressor binding sites (FIGS. 2 and 6). Surprisingly, the specific disruption of a single repressor binding site, in particular Let7c binding site, increased utrophin expression as efficiently as the deletion of the complete region of the repressor binding site comprising a cluster of repressor binding sites (FIG. 4).

Thus, the present disclosure relates to a method for enhancing utrophin expression in cell comprising introducing into a cell a composition comprising at least one gene editing enzyme capable of inducing site-specific mutations within a target sequence comprising at least one repressor binding site of utrophin gene selected from the group consisting of: Ets-2-repressor factor (ERF) binding sites, homeobox protein engrailed-1 (EN1) binding site 2, Let7c binding site and miR-196b binding site and wherein said site-specific mutation disrupts the repressor binding without deleting the whole repressor binding site sequence.

The present disclosure also relates to a composition for enhancing utrophin expression in cell, comprising a gene editing enzyme capable of inducing site-specific mutations within a target sequence comprising a repressor binding site of utrophin gene selected from the group consisting of: Ets-2-repressor factor (ERF) binding sites, homeobox protein engrailed-1 (EN1) binding site 2, Let7c binding site and miR196b binding site and wherein the mutations disrupt the repressor binding site without deleting the whole repressor binding site sequence. The mutation(s) introduced in these specific target sequences inhibit repressor binding on utrophin (UTRN) gene or mRNA and consequently UTRN expression is increased.

As used herein “site-specific mutation(s) which disrupt the repressor binding site” refers to mutation(s) which modify a part of the repressor binding site sequence without deleting the whole repressor binding site sequence. The mutations which disrupt the repressor binding site alter the binding site in a way which inhibits repressor binding on utrophin gene or mRNA. As used herein ‘inhibits’’ refers to a partial or total inhibition. The inhibition of repressor binding on utrophin gene or mRNA induces an increase of utrophin expression.

Human utrophin (UTRN) gene (Gene ID: 7402 ; Ensembl:ENSG00000152818 MIM:128240) is on chromosome 6 and comprises multiple small exons spanning approximately 900 kb and a long 5′ untranslated region composed of 2 exons. The utrophin mRNA contains two full-length species (named A- and B-utrophin), which have different initial exons and are transcribed from different promoters. The predicted protein sequences arising from these transcripts differ at their N termini with unique sections of 31 and 26 amino acids, respectively. Hence, utrophin is a composite of A- and B-utrophin and only the A-utrophin is up-regulated in dystrophin-deficient striated muscle. The UTRN gene is conserved in chimpanzee, Rhesus monkey, dog, cow, mouse, rat, chicken, zebrafish, and frog. Human UTRN orthologs are found in many organisms.

As used herein, the singular forms “a”, “an”, and “the” include both singular and plural referents unless the context clearly dictates otherwise.

The regulation of utrophin expression is complex. Although, several upregulation elements have been identified in the promoter, utrophin mRNA is also subject to transcriptional or translational repression mediated by its 5′- and 3′-UTR regions. Several regulatory sequences involved in the repression of utrophin expression in adult muscle, herein called utrophin repressor binding site have been identified. Utrophin repressor binding sites include as non-limiting examples, sequences within 3′-UTR region of utrophin gene such as AU-rich elements (AREs) as described in Amirouche A. et al. Hum. Mol. Genet. 2013, 22(15):3093-3111 and Gramolini A. O. et al J. cell. Biol. 2001, 154:1173-83 or sequences targeted by miRNA, preferably by let7c, miR-296-5p (I), miR206, and miR-196b binding sites; or sequences within 5′UTR/promoter-enhancer region of utrophin gene such as the Ets-2-repressor factor (ERF) binding site, also named N/box-EBS site, or binding site 1 or 2 for homeobox protein engrailed-1 (EN1).

The inventors showed that the disruption of specific repressor binding sites selected from the group consisting of Let7c binding site, miR-196b binding site, ERF binding site and EN1 binding site 2 is efficient to increase utrophin expression.

In a preferred embodiment, said repressor binding site may be in the 3′UTR sequence of utrophin gene localized from positions 144,850,989 to 144,853,034 of chromosome 6, GRCh38.p13 (genome reference consortium (March 2019), Ref Seq CGF_000001405.39) and said repressor binding site is let7c binding site of SEQ ID NO: 1 (positions 144,852,607 to 144,852,626 of chromosome 6, GRCh38.p13 (genome reference consortium (March 2019), Ref Seq CGF_000001405.39) or miR-196b binding site of SEQ ID NO: 2.

In another preferred embodiment, said repressor binding site may be in the 5′UTR sequence of utrophin gene localized upstream of utrophin A exon 1 which starts at 144,291,829 of chromosome 6, GRCh38.p13 (genome reference consortium (March 2019), Ref Seq CGF_000001405.39) and said repressor binding site is Ets-2 repressor (ERF) factor binding site, also named N/box-EBS site localized 144,285,022 to 144,285,026 of chromosome 6 GRCh38.p13 (genome reference consortium (March 2019), Ref Seq CGF_000001405.39) consisting of sequence CGGAA or homeobox protein engrailed-1 (EN1) binding site 2 localized 144,285,004 to 144,285,010 of chromosome 6 GRCh38.p13 (genome reference consortium (March 2019), Ref Seq CGF_000001405.39) consisting of sequence GTAGTGG.

In some preferred embodiments, said repressor binding site is let7c binding site; preferably consisting of SEQ ID NO: 1.

Advantageously, the disruption of repressor binding site within 5′UTR/promoter-enhancer region of utrophin gene allows to increase specifically transcription of utrophin A, which is up-regulated in dystrophin-deficient striated muscle.

The sequences of a number of different mammalians utrophin repressor binding sites are known including, but being not limited to, human, pig, chimpanzee, dog, cow, mouse, rabbit or rat, and can be easily found in sequence databases. In some preferred embodiments, said utrophin gene is human.

According to the present disclosure, the inventors used gene editing enzyme to specifically induce site-specific mutation(s) within a target sequence comprising a utrophin repressor binding site. Gene editing enzyme may be sequence-specific nuclease, base or prime editor.

In a particular embodiment, said gene editing enzyme is a sequence-specific nuclease.

The term “nuclease” refers to a wild type or variant enzyme capable of catalyzing the hydrolysis (cleavage) of phosphodiester bonds between nucleotides of a nucleic acid (DNA or RNA) molecule, preferably a DNA molecule. By “cleavage” is intended a double-strand break or a single-strand break event.

The term “sequence-specific nuclease” refers to a nuclease which cleaves nucleic acid in a sequence-specific manner. Different types of site-specific nucleases can be used, such as Meganucleases, TAL-nucleases (TALEN), Zing-finger nucleases (ZFN), or RNA/DNA guided endonucleases like Clustered Regularly Interspaced Short Palindromic Repeats (CRISPR)/Cas system and Argonaute (Review in Li et al., Nature Signal transduction and targeted Therapy, 5, 2020; Guha et al., Computational and Structural Biotechnology Journal, 2017, 15, 146-160).

According to the present disclosure, the nuclease generates a nucleic acid cleavage, preferably a DNA cleavage, within the target sequence by sequence-specific targeting of the sequence comprising a utrophin repressor binding site. By “sequence-specific targeting of the sequence comprising a repressor binding site”, it is intended targeting a part of the sequence comprising the repressor binding site as described above and/or sequences adjacent to said repressor binding site, in particular at least one (one or two) sequence of up to 15 nucleotides adjacent to said repressor binding site, preferably 10, 9, 8, 7, 6 or 5 nucleotides adjacent to said repressor binding site.

According to the present disclosure, the target sequence comprises or consists of the portion sequence from the nucleotide in position -15 to the nucleotide in position +15, relative to the 5′- and 3′-end respectively of the utrophin repressor binding site sequence as disclosed herein.

Said target sequences comprising utrophin repressor binding sites are represented in the Table 1 below.

TABLE 1

Target sequences comprising utrophin repressor binding sites (repressor binding sites are underlined)

| UTRN repressor binding site | Target sequence of UTRN repressor binding site sequence | SEQ ID NO |
|---|---|---|
| miR-Let7c binding site | TTATATAAAAAGGAAAGCCATGACCACCTTTCTACCTCA GATCCATCTTCAT | 18 |
| | TTATATAAAAAGGAAAGCCATGACCACCTTTCTACCTCA GATCCATCTTCATCC | 26 |
| miR-196b | TTTTATCAGGCCATGTCATACCCAAGAAAGCACCT ATTTAAAGAAAAAAC | 19 |
| ERF binding site | AGTGGGGCTGATCTTCCGGAA CAAAGTTGCTGGGCC | 20 |
| EN1 binding site 2 | CGCTGACCCGGGAACGTAGTGG GGCTGATCTTCCGGA | 21 |

In particular, the target sequence comprising the Let7c binding site is SEQ ID NO: 18 or SEQ ID NO: 26, the target sequence comprising mir-196b binding site is SEQ ID NO: 19, the target sequence comprising ERF binding site is SEQ ID NO: 20 and the target sequence comprising the EN1 binding site 2 is SEQ ID NO: 21.

As disclosed herein, the cleavage of UTRN gene target sequence induces site specific mutations, particularly indel and/or substitution mutations in the target sequence which disrupt said repressor binding site and thereby increases UTRN expression by inhibiting UTRN repressor binding on UTRN gene or mRNA.

The DNA strand break that is introduced by the nuclease according to the invention is repaired by cell’s own DNA repair processes such as non-homologous (NHEJ) and microhomology mediated (MMEJ) end joining pathways which induce small insertion and deletions (indels) and substitutions.

The term “indels” refers to insertion deletion mutagenic events resulting from cell’s own DNA repair mechanism such as NHEJ or MMEJ following the introduction of a DNA cleavage within a target sequence comprising a utrophin repressor binding site using a sequence-specific nuclease according to the present disclosure. According to the present disclosure, said indels occur in a target sequence comprising a utrophin repressor binding site and inhibit the function of this element, in particular repression of the transcription or translation of utrophin gene. In another term, said indels induce an increase of utrophin gene expression level. As used herein indels within a target sequence comprising a repressor binding site according to the present disclosure are different from the deletion of the repressor binding site induced by two site-specific nucleases targeting sequences upstream and downstream of the repressor binding site as disclosed in the prior art.

In some embodiments, indels refer to insertion deletion mutagenic events where no more than 50 nucleotide bases are changed, inserted and/or deleted from DNA or RNA sequence. The size of the indels depends on the gene editing enzyme. For example, for SpCas9, single-nucleotides are the most frequent type of indel with the majority of targets showing 1-nt insertion or deletion, respectively as the commonest indel. Nevertheless, sites showing a preference for longer deletions (for example up to 41 nt) may be observed (Chakrabarti et al., Molecular Cell, 2019, 73, 699-713; Kurgan et al., Molecular Therapy: Methods & Clinical Development; 2021, 21, 478-491).

According to the present disclosure, said sequence-specific nuclease cleaves and induces site-specific mutations within a target sequence comprising an utrophin repressor binding site to inhibit repressor binding on utrophin gene or mRNA and thereby increase UTRN gene expression.

In particular embodiments, the inventors used CRISPR system to induce a cleavage within a target sequence comprising a utrophin repressor binding site as described above.

CRISPR system involves two components, Cas protein (CRISPR-associated protein) and single guide RNA. Cas protein is a DNA endonuclease that uses guide RNA sequence as a guide to recognize and generate double-strand cleavage in DNA that is complementary to the single guide RNA sequence. Cas protein comprises two active cutting sites namely HNH nuclease domain and RuvC-like nuclease domain.

By Cas protein is also meant an engineered endonuclease or a homologue of Cas 9 which is capable of cleaving target nucleic acid sequence. In particular embodiments, Cas protein may induce a cleavage in the nucleic acid target sequence which can correspond to either a double-stranded break or a single- stranded break. Cas protein variant may be a Cas endonuclease that does not naturally exist in nature and that is obtained by protein engineering or by random mutagenesis. The Cas protein can be one type of the Cas proteins known in the art. Non-limiting examples of Cas proteins include Cas1, Cas1B, Cas2, Cas3, Cas4, Cas5, Cas6, Cas7, Cas8, Cas9 (also known as Csn1 and Csx12), Cas12 (Cas12a or Cpf1), Cas10, Csy1, Csy2, Csy3, Cse1, Cse2, Csc1, Csc2, Csa5, Csn2, Csm2, Csm3, Csm4, Csm5, Cmr1, Cmr3, Cmr4, Cmr5, Cnrr6, Csb1, Csb2, Csb3, Csx17, CsxM, Csx 10, Cs 16, CsaX, Csx3, Cs 1, Csx15, Csf1, Csf2, Cs0, Csf4, homologs, orthologs thereof, or modified versions thereof. Preferably Cas protein is Streptococcus pyogenes Cas 9 protein and orthologs thereof such as Staphylococcus aureus Cas9 protein and Streptococcus thermophilus Cas9. Another preferred Cas protein is Cas12a, such as Cas12a from Acidaminococccus or Lachnospiracae. Cas12a variants with enhanced activity are disclosed in Liyang Zhang et al. (Nature Communications, 2021, doi: 10.1038).

Cas is contacted with a guide RNA (gRNA) designed to comprise a complementary sequence of the target nucleic acid sequence to specifically induce DNA cleavage within said target sequence, in particular according to the present disclosure a complementary sequence of a part of said target sequence comprising utrophin repressor binding site as described above.

As used herein, a “guide RNA”, “gRNA” or “single guide RNA” refers to a nucleic acid that promotes the specific targeting or homing of a gRNA/Cas complex to a target nucleic acid.

In particular, gRNA refers to a RNA that comprises a transactivating crRNA (tracrRNA) and a crRNA. Preferably, said guide RNA corresponds to a crRNA and tracrRNA which can be used separately or fused together. The complementary sequence pairing with the target sequence recruits Cas to bind and cleave the DNA at the target sequence.

According to the present disclosure, crRNA is engineered to comprise a complementary sequence to a part of a target sequence comprising an utrophin repressor binding site, such that it is capable of targeting said region. By “targeting repressor binding site” it is intended targeting at least a part of a sequence comprising a repressor binding site as described above and/or sequence(s) adjacent to said repressor binding site, in particular at least a sequence(s) of up to 15 nucleotides adjacent to said repression binding site, preferably 10, 9, 8, 7, 6 or 5 nucleotides adjacent to said repressor binding site.

In a particular embodiment, the crRNA comprises a sequence of 5 to 50 nucleotides, preferably 15 to 30 nucleotides, more preferably 20 nucleotides which is complementary to the target sequence. According to the present disclosure said target sequence is a DNA sequence comprising the utrophin repressor binding site as described above and adjacent to protospacer adjacent motif (PAM).

As used herein, the terms “complementary sequence” refers to the sequence part of a polynucleotide (e.g. part of crRNA or tracRNA) that can hybridize to another part of polynucleotides under standard low stringent conditions. Preferentially, the sequences are complementary to each other pursuant to the complementarity between two nucleic acid strands relying on Watson-Crick base pairing between the strands, i.e. the inherent base pairing between adenine and thymine (A-T) nucleotides and guanine and cytosine (G-C) nucleotides.

Said gRNA can be designed by any methods known by one of skill in the art in view of the present disclosure. In a particular embodiment, said gRNA may target utrophin repressor binding site as described above and comprise one of the sequences described in the Table 1 (gRNA sequence).

TABLE 2

UTRN repressor binding site sequence and gRNA sequences used to target the corresponding UTRN repressor binding site

| UTRN repressor binding site | Target sequence of UTRN repressor binding site sequence | SEQ ID NO | gRNA sequence | SEQ ID NO |
|---|---|---|---|---|
| miR-Let7c binding site | AGCCATGACCACCTTTCTACCTCA | 1 | CTGAGGTAGAAAGGTGATCA | 3 |
| | | | CTGAGGTAGAAAGGTGGTCA | 4 |
| | | | ATGGATCTGAGGTAGAAAGG | 5 |
| | | | AAGATGGATCTGAGGTAGAA | 6 |
| | | | AAGGTGGTTCTGAGGTAGAA | 25 |
| miR-196b binding site | TCATACCCAAGAAAGCACCT | 2 | GTGCTTTCTTGGGTATGACA | 7 |
| | | | CTTTAAATAGGTGCTTTCTT | 8 |
| ERF binding site | CGGAA | | TCTTCCGGAACAAAGTTGCT | 9 |
| | | | GAACAAAGTTGCTGGGCCGG | 10 |
| | | | ACGTAGTGGGGCTGATCTTC | 11 |
| | | | CCGGCCCAGCAACTTTGTTC | 12 |
| | | | ATCTTCCGGAACAAAGTTGC | 13 |
| | | | TCTTCCGGAACAAAGTTGCT | 14 |
| EN1 binding site 2 | GTAGTGG | | ATCAGCCCCACTACGTTCCC | 15 |
| | | | GCTGACCCGGGAACGTAGTG | 16 |
| | | | ACGCTGACCCGGGAACGTAG | 17 |

SEQ ID NO: 1 and 2 correspond to the (+) strand of the UTRN repressor binding site. The gRNA sequences SEQ ID NO: 3, 4, 5, 6, 7, 8, 12, 15 and 25 correspond to the (-) strand of the UTRN repressor binding site. The gRNA sequences SEQ ID NO: 9, 10, 11, 13, 14, 16 and 17 correspond to the (+) strand of the UTRN repressor binding site.

The gRNA sequences presented in Table 2 are indicated in the form of DNA sequences corresponding to the gRNA molecule, which means the DNA equivalent of the (RNA) sequence of the gRNA.
CTGAGGTAGAAAGGTGATCA (SEQ ID NO: 3) corresponds to the gRNA having the sequence CUGAGGUAGAAAGGUGAUCA (SEQ ID NO: 27).
CTGAGGTAGAAAGGTGGTCA (SEQ ID NO: 4) corresponds to the gRNA having the sequence CUGAGGUAGAAAGGUGGUCA (SEQ ID NO: 28).
ATGGATCTGAGGTAGAAAGG (SEQ ID NO: 5) corresponds to the gRNA having the sequence AUGGAUCUGAGGUAGAAAGG (SEQ ID NO: 29).
AAGATGGATCTGAGGTAGAA (SEQ ID NO: 6) corresponds to the gRNA having the sequence AAGAUGGAUCUGAGGUAGAA (SEQ ID NO: 30).
AAGGTGGTTCTGAGGTAGAA (SEQ ID NO: 25) corresponds to the gRNA having the sequence AAGGUGGUUCUGAGGUAGAA (SEQ ID NO: 31).
GTGCTTTCTTGGGTATGACA (SEQ ID NO: 7) corresponds to the gRNA having the sequence GUGCUUUCUUGGGUAUGACA (SEQ ID NO: 32).
CTTTAAATAGGTGCTTTCTT (SEQ ID NO: 8) corresponds to the gRNA having the sequence CUUUAAAUAGGUGCUUUCUU (SEQ ID NO: 33).
TCTTCCGGAACAAAGTTGCT (SEQ ID NO: 9) corresponds to the gRNA having the sequence UCUUCCGGAACAAAGUUGCU (SEQ ID NO: 34).
GAACAAAGTTGCTGGGCCGG (SEQ ID NO: 10) corresponds to the gRNA having the sequence GAACAAAGUUGCUGGGCCGG (SEQ ID NO: 35).
ACGTAGTGGGGCTGATCTTC (SEQ ID NO: 11) corresponds to the gRNA having the sequence ACGUAGUGGGGCUGAUCUUC (SEQ ID NO: 36).
CCGGCCCAGCAACTTTGTTC (SEQ ID NO: 12) corresponds to the gRNA having the sequence CCGGCCCAGCAACUUUGUUC (SEQ ID NO: 37).
ATCTTCCGGAACAAAGTTGC (SEQ ID NO: 13) corresponds to the gRNA having the sequence AUCUUCCGGAACAAAGUUGC (SEQ ID NO: 38).
TCTTCCGGAACAAAGTTGCT (SEQ ID NO: 14) corresponds to the gRNA having the sequence UCUUCCGGAACAAAGUUGCU (SEQ ID NO: 39).
ATCAGCCCCACTACGTTCCC (SEQ ID NO: 15) corresponds to the gRNA having the sequence AUCAGCCCCACUACGUUCCC (SEQ ID NO: 40).
GCTGACCCGGGAACGTAGTG (SEQ ID NO: 16) corresponds to the gRNA having the sequence GCUGACCCGGGAACGUAGUG (SEQ ID NO: 41).
ACGCTGACCCGGGAACGTAG (SEQ ID NO: 17) corresponds to the gRNA having the sequence ACGCUGACCCGGGAACGUAG (SEQ ID NO: 42).

The present disclosure encompasses gRNA variants targeting utrophin repressor binding site, which differ from the above gRNA sequences by up to 5 (1, 2, 3, 4 or 5) mutations (substitution, deletion or insertion).

The present disclosure encompasses chemically modified gRNAs, in particular gRNA comprising at least one chemical modification that improves editing. Chemical modifications of gRNA that improve editing, in particular in most cell types, including primary cells and stem cells in vitro and in vivo are well-known in the art (see for example Allen et al., Front. Genome Ed., 28 Jan. 2021, doi: 10.3389). Non limiting examples include 2′-O-Methyl at 3 first and last bases and 3′ phosphorothionate bonds between first 3 and last 2 bases of gRNA.

In a particular embodiment said gRNA may target miR-let7c binding site and comprises a sequence selected from the group consisting of SEQ ID NO: 3 to 6 or SEQ ID NO: 3 to 6 and 25. In a particular embodiment said gRNA may target miR-196-b binding site and comprises a sequence of SEQ ID NO: 7 or 8, preferably SEQ ID NO: 7. In a particular embodiment, said gRNA may target ERF binding site and comprises a sequence of SEQ ID NO: 9 or 14, preferably SEQ ID NO: 9. In a particular embodiment, said gRNA may target EN1 binding site 2 and comprises a sequence of SEQ ID NO: 15 to 17; preferably SEQ ID NO: 16. In some preferred embodiments, said gRNA targets miR-let7c binding site, ERF binding site or EN1 binding site 2; preferably said gRNA comprises a sequence selected from SEQ ID NO: 3 to 6, 9, 16 and 25. In other preferred embodiments, said gRNA targets miR-let7cbinding site; preferably said gRNA comprises a sequence selected from the group consisting of SEQ ID NO: 3 to 6 and 25; preferably SEQ ID NO: 5.

In another particular embodiment, said gene editing enzyme is a DNA base editor as described in Komor et al., Nature 533, 420-424, doi:10.1038/nature17946 and in Rees HA, Liu DR. Nat Rev Genet. 2018 Dec;19(12):770-788 or a prime editor as described in Anzalone AV. Et al. Nature, 2019, 576:149-157, Matsoukas IG. Front Genet. 2020; 11: 528 and Kantor A. et al. Int. J. Mol. Sci. 2020, 21(6240).

The use of base editor or prime editor allows the introduction of mutations, preferably point mutations at specific sites in the target sequence.

According to the present disclosure, the base editor or prime editor generates a mutation within the target sequence by sequence-specific targeting of the sequence comprising a utrophin repressor binding site. By “sequence-specific targeting of the sequence comprising a repressor binding site”, it is intended targeting a part of the sequence comprising the repressor binding site as described above and/or sequences adjacent to said repressor binding site, in particular at least one (one or two) sequence of up to 15 nucleotides adjacent to said repressor binding site, preferably 10, 9, 8, 7, 6 or 5 nucleotides adjacent to said repressor binding site.

Said base editor consists of a fusion of a catalytically inactive sequence specific nuclease as described above that is capable to target a specific DNA target sequence and a catalytically active base modification enzyme, such as a nucleotide deaminase domain.

In particular, said base editor or prime editor are CRISPR base or prime editor. Said CRISPR base or prime editor comprises as catalytically inactive sequence specific nuclease a dead Cas protein (dCas). dCas refers to a modified Cas nuclease which lacks endonucleolytic activity. Nuclease activity can be inhibited or prevented in dCas proteins by one or more mutations and/or one or more deletions in the HNH and/or RuvC-like catalytic domains of the Cas protein. The resulting dCas protein lacks nuclease activity but bind to a guide RNA (gRNA)-DNA complex with high specificity and efficiency to specific target sequence. In particular embodiment, said dead Cas may be a Cas nickase wherein one catalytic domain of the Cas is inhibited or prevented.

Said base editor is contacted with a guide RNA (gRNA) designed to comprise a complementary sequence of the target nucleic acid sequence to specifically bind said target sequence, in particular according to the present disclosure a complementary sequence of a part of said target sequence comprising utrophin repressor binding site as described above.

Said gRNA can be designed by any methods known by one of skill in the art in view of the present disclosure. In a particular embodiment, said gRNA may target utrophin repressor binding site as described above and comprises one of the sequences described in the Table 2 (gRNA sequence).

As non-limiting examples said base editor is a nucleotide deaminase domain fused to a dead Cas protein, in particular Cas nickase. Said nucleotide deaminase may be an adenosine deaminase or cytidine deaminase.

In a particular embodiment, said base editor may be as non-limiting examples selected from the group consisting of: BE1, BE2, BE3, BE4, HF-BE3, Sa-BE3, Sa-BE4, BE4-Gam, saBE4-Gam, YE1-BE3, EE-BE3, YE2-BE3, YEE-BE3, VQR-BE3, VRER-BE3, SaKKH-BE3, cas12a-BE, Target-AID, Target-AID-NG, xBE3, eA3A-BE3, A3A-BE3, BE-PLUS, TAM, CRIPS-X, ABE7.9, ABE7.10, ABE7.10* xABE, ABESa, VQR-ABE, VRER-ABE and SaKKH-ABE.

Said prime editor consists of a fusion of a catalytically inactive sequence specific nuclease as described above, particularly a Cas nickase or a wild-type Cas and a catalytically active engineered reverse transcriptase (RT) enzyme. Said fusion protein is used in combination with a prime editing guide RNA (pegRNA) which contains the complementary sequence to the target sequence as described above, particularly comprises one of the sequences described in the Table 2 and also an additional sequence comprising a sequence that binds to the primer binding site region on the DNA. In particular embodiment, said reverse transcriptase enzyme is a Maloney murine leukemia virus RT enzyme and variants thereof. Said prime editor may be as non-limiting examples selected from the group consisting of: PE1, PE2, PE3 and PE3b.

The composition according to the present disclosure increases utrophin expression in cell in vitro and/or in vivo; preferably in cell expressing dystrophin such as muscle cells.

The utrophin gene expression is enhanced in cells when the expression level of the utrophin gene is at least 1.5-fold higher, or 2, 3, 4, 5-fold higher in cells treated with the gene editing enzyme than in untreated cells. The increase of utrophin gene expression which may be at the RNA or protein level may be determined by any suitable methods known by skilled persons.

For example, the nucleic acid contained in the sample is first extracted according to standard methods, for example using lytic enzymes or chemical solutions or extracted by nucleic-acid-binding resins following the manufacturer’s instructions. The level of UTRN mRNA is then detected by hybridization (e.g., Northern blot analysis) and/or amplification (e.g., RT-PCR).

The level of UTRN protein may also be determined by any suitable methods known by skilled persons. The quantity of the protein may be measured, for example, by semiquantitative Western blots, enzyme-labelled and mediated immunoassays, such as ELISAs, biotin/avidin type assays, radioimmunoassay, immunoelectrophoresis, mass spectrometry, or immunoprecipitation or by protein or antibody arrays.

Said gene editing enzyme such as gRNA and Cas protein can be constructed using chemical synthesis and enzymatic ligation reactions using procedures known in the art and can be delivered to a cell using any-known techniques including but being not limited to calcium phosphate transfection, DEAE-Dextran transfection, electroporation, microinjection, biolistic, viral infection or liposome-mediated transfection.

In some embodiments, the composition for enhancing utrophin expression comprises a single sequence-specific nuclease capable of inducing a single mutation event within each target sequence comprising a utrophin repressor binding site.

In another particular embodiment, said composition for enhancing utrophin expression may comprise at least two gene editing enzymes as described above capable of inducing mutation events within one or more target sequences comprising a utrophin repressor binding site.

In particular, when said gene editing enzymes are sequence-specific nucleases, said sequence-specific nucleases can be used successively in such a way that a first sequence-specific nuclease cleaves and induces a first mutation event within a target sequence. Once first mutation event is repaired, a second sequence-specific nuclease may be used to cleave and induce a second mutation event within said or another target sequence.

In another particular embodiment, when said at least two gene editing enzymes are base or prime editors, said gene editing enzymes can be used simultaneously. In another particular embodiment, when said two gene editing enzymes are a base or prime editor and a single sequence-specific nuclease, said gene editing enzymes can also be used simultaneously.

In some preferred embodiments, the target sequences of the at least two gene editing enzymes are different. In some embodiments, the composition for enhancing utrophin expression comprises a gene editing enzyme capable of inducing sequence-specific mutation(s) within a target sequence consisting of a utrophin repressor binding site.

### Nucleic Acid Construct and Expression Vector

In one embodiment said gene editing enzyme are encoded by one or more nucleic acid constructs.

The term “nucleic acid construct” as used herein refers to a man-made nucleic acid molecule resulting from the use of recombinant DNA technology. A nucleic acid construct is a nucleic acid molecule, either single- or double-stranded, which has been modified to contain segments of nucleic acid sequences, which are combined and juxtaposed in a manner, which would not otherwise exist in nature. A nucleic acid construct usually is a “vector”, i.e. a nucleic acid molecule which is used to deliver exogenously created DNA into a host cell.

Preferably, the nucleic acid construct comprises said gene editing enzyme, operably linked to one or more control sequences that direct the expression in muscle cells.

Said control sequences may be a ubiquitous, tissue-specific or inducible promoter which is functional in cells of target organs (i.e. muscles). Such sequences which are well-known in the art include in particular a promoter, and further regulatory sequences capable of further controlling the expression of a transgene, such as without limitation, enhancer, terminator, intron, silencer, in particular tissue-specific silencer, and microRNA.

Examples of ubiquitous promoters include the CAG promoter, phosphoglycerate kinase 1 (PGK) promoter, the cytomegalovirus enhancer/promoter (CMV), the SV40 early promoter, the retroviral Rous sarcoma virus (RSV) LTR promoter, the dihydrofolate reductase promoter, the β-actin promoter, and the EF1 promoter.

Muscle-specific promoters include without limitation, the desmin (Des) promoter, muscle creatine kinase (MCK) promoter, CK6 promoter, alpha-myosin heavy chain (alpha-MHC) promoter, myosin light chain 2 (MLC-2) promoter, cardiac troponin C (cTnC) promoter, synthetic muscle-specific SpC5-12 promoter, the human skeletal actin (HSA) promoter.

In a preferred embodiment, said nucleic acid construct comprises gene editing enzyme capable of targeting utrophin repressor binding site region comprising a sequence selected from the group consisting of: sequence CGGAA, GTAGTGG, SEQ ID NO: 1 and 2.

In a more preferred embodiment, said nucleic acid construct comprises a gene editing enzyme capable of targeting let7c binding site comprising a gRNA sequence selected from the group consisting of: SEQ ID NO: 3 to 6 or SEQ ID NO: 3 to 6 and 25, preferably SEQ ID NO: 5.

In another preferred embodiment, said nucleic acid construct comprises gene editing enzyme capable of targeting miR196-b binding site comprising a gRNA sequence selected from the group consisting of: SEQ ID NO: 7 or 8, preferably SEQ ID NO: 7.

In another preferred embodiment, said nucleic acid construct comprises gene editing enzyme capable of targeting ERF binding site comprising a gRNA sequence selected from the group consisting of: SEQ ID NO: 9 to 14, preferably SEQ ID NO: 9.

In another preferred embodiment, said nucleic acid construct comprises gene editing enzyme capable of targeting EN1 binding site 2 comprising a gRNA sequence selected from the group consisting of: SEQ ID NO: 15 to 17; preferably SEQ ID NO: 16.

The nucleic acid construct as described above may be contained in an expression vector. The vector may be an autonomously replicating vector, i.e., a vector that exists as an extra-chromosomal entity, the replication of which is independent of chromosomal replication, e.g., a plasmid, an extra-chromosomal element, a mini-chromosome, or an artificial chromosome. The vector may contain any means for assuring self-replication. Alternatively, the vector may be one that, when introduced into the host cell, is integrated into the genome and replicated together with the chromosome(s) into which it has been integrated.

Examples of appropriate vectors include, but are not limited to, recombinant integrating or non- integrating viral vectors and vectors derived from recombinant bacteriophage DNA, plasmid DNA or cosmid DNA. Preferably, the vector is a recombinant integrating or non-integrating viral vector. Examples of recombinant viral vectors include, but not limited to, vectors derived from herpes virus, retroviruses, lentivirus, vaccinia viruses, adenoviruses, adeno-associated viruses or bovine papilloma virus.

AAV has arisen considerable interest as a potential vector for human gene therapy. Among the favourable properties of the virus are its lack of association with any human disease, its ability to infect both dividing and non-dividing cells, and the wide range of cell lines derived from different tissues that can be infected.

The AAV genome is composed of a linear, single-stranded DNA molecule which contains 4681 bases (Berns and Bohenzky, 1987, Advances in Virus Research (Academic Press, Inc.) 32:243-307). The genome includes inverted terminal repeats (ITRs) at each end, which function in cis as origins of DNA replication and as packaging signals for the virus. The ITRs are approximately 145 bp in length. The internal non-repeated portion of the genome includes two large open reading frames, known as the AAV rep and cap genes, respectively. These genes code for the viral proteins involved in replication and packaging of the virion.

In particular, at least four viral proteins are synthesized from the AAV rep gene, Rep 78, Rep 68, Rep 52 and Rep 40, named according to their apparent molecular weight. The AAV cap gene encodes at least three proteins, VP1, VP2 and VP3. For a detailed description of the AAV genome, see, e.g., Muzyczka, N. 1992 Current Topics in Microbiol. and Immunol. 158:97-129.

The present disclosure relates to an AAV vector comprising guide RNA and/or Cas protein as described above.

Thus, in one embodiment, the nucleic acid construct or expression vector comprising guide RNA and/or Cas protein as described above further comprises a 5′ITR and a 3′ITR sequences, preferably a 5′ITR and a 3′ ITR sequences of an adeno-associated virus.

As used herein the term “inverted terminal repeat (ITR)” refers to a nucleotide sequence located at the 5′-end (5′ITR) and a nucleotide sequence located at the 3′-end (3′ITR) of a virus, that contain palindromic sequences and that can fold over to form T-shaped hairpin structures that function as primers during initiation of DNA replication. They are also needed for viral genome integration into the host genome; for the rescue from the host genome; and for the encapsidation of viral nucleic acid into mature virions. The ITRs are required in cis for the vector genome replication and its packaging into the viral particles.

AAV ITRs for use in the viral vector of the disclosure may have a wild-type nucleotide sequence or may be altered by the insertion, deletion or substitution. The serotype of the inverted terminal repeats (ITRs) of the AAV may be selected from any known human or nonhuman AAV serotype. In specific embodiments, the nucleic acid construct or viral expression vector may be carried out by using ITRs of any AAV serotype, including AAV1, AAV2, AAV3 (including types 3A and 3B), AAV4, AAV5, AAV6, AAV7, AAV8, AAV9, AAVrh10, AAV11, AAV12, avian AAV, bovine AAV, canine AAV, equine AAV, ovine AAV, and any other AAV serotype or engineered AAV now known or later discovered.

In one embodiment, the nucleic acid construct comprises a 5′ITR and a 3′ITR of the corresponding capsid, or preferably 5′ITR and a 3′ITR of a serotype AAV-2.

On the other hand, the nucleic acid construct or expression vector of the disclosure can be carried out by using synthetic 5′ITR and/or 3′ITR; and also by using a 5′ITR and a 3′ITR which come from viruses of different serotypes. All other viral genes required for viral vector replication can be provided in trans within the virus-producing cells (packaging cells) as described below. Therefore, their inclusion in the viral vector is optional.

In one embodiment, the nucleic acid construct or viral vector of the disclosure comprises a 5′ITR, a ψ packaging signal, and a 3′ITR of a virus. “ψ packaging signal” is a cis-acting nucleotide sequence of the virus genome, which in some viruses (e.g. adenoviruses, lentiviruses ...) is essential for the process of packaging the virus genome into the viral capsid during replication.

The construction of recombinant AAV viral particles is generally known in the art and has been described for instance in US 5,173,414 and US5,139,941; WO 92/01070, WO 93/03769, Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Current Topics in Microbiol. and Immunol. 158:97-129; and Kotin, R. M. (1994) Human Gene Therapy 5:793-801.

### Viral Particle

In a preferred embodiment, the present disclosure relates to viral particles including a nucleic acid construct or expression vector as described above.

The nucleic acid construct or the expression vector of the disclosure may be packaged into a virus capsid to generate a “viral particle”, also named “viral vector particle”. In a particular embodiment, the nucleic acid construct or the expression vector as described above is packaged into an AAV-derived capsid to generate an “adeno-associated viral particle” or “AAV particle”. The present disclosure relates to a viral particle comprising a nucleic acid construct or an expression vector of the disclosure and preferably comprising capsid proteins of adeno-associated virus.

The term AAV vector particle encompasses any recombinant AAV vector particle or mutant AAV vector particle, genetically engineered. A recombinant AAV particle may be prepared by encapsidating the nucleic acid construct or viral expression vector including ITR(s) derived from a particular AAV serotype on a viral particle formed by natural or mutant Cap proteins corresponding to an AAV of the same or different serotype.

Proteins of the viral capsid of an adeno-associated virus include the capsid proteins VP1, VP2, and VP3. Differences among the capsid protein sequences of the various AAV serotypes result in the use of different cell surface receptors for cell entry. In combination with alternative intracellular processing pathways, this gives rise to distinct tissue tropisms for each AAV serotype.

Several techniques have been developed to modify and improve the structural and functional properties of naturally occurring AAV viral particles (Bünning H et al. J Gene Med, 2008; 10: 717-733; Paulk et al. Mol ther. 2018; 26(1):289-303; Wang L et al. Mol Ther. 2015; 23(12):1877-87; Vercauteren et al. Mol Ther. 2016; 24(6):1042-1049; Zinn E et al., Cell Rep. 2015; 12(6):1056-68).

Thus, in AAV viral particle according to the present disclosure, the nucleic acid construct or viral expression vector including ITR(s) of a given AAV serotype can be packaged, for example, into: a) a viral particle constituted of capsid proteins derived from the same or different AAV serotype; b) a mosaic viral particle constituted of a mixture of capsid proteins from different AAV serotypes or mutants; c) a chimeric viral particle constituted of capsid proteins that have been truncated by domain swapping between different AAV serotypes or variants.

The skilled person will appreciate that the AAV viral particle for use according to the present disclosure may comprise capsid proteins from any AAV serotype including AAV1, AAV2, AAV3 (including types 3A and 3B), AAV4, AAV5, AAV6, AAV7, AAV8, AAV9, AAV10, AAV11, AAV12, AAV2i8, AAVrh10, AAVrh39, AAVrh43, AAVrh74, AAV-LK03, AAV2G9, AAV.PHP, AAV-Anc80, AAV3B and AAV9.rh74 (as disclosed in WO2019/193119).

For gene transfer into muscle, AAV serotypes 2, 6, 8, 9, AAVrh10 or AAV9.rh74 are preferred. In a specific embodiment, the AAV viral particle comprises a nucleic acid construct or expression vector of the disclosure and preferably capsid proteins from AAV9 or AAV9.rh74 serotype.

### Method for Enhancing Utrophin Expression in a Cell

The present disclosure relates to a method for enhancing utrophin expression in a cell by inducing site-specific mutation(s) within a target sequence comprising a repressor binding site of utrophin gene selected from the group consisting of: Let7c binding site, miR-196b binding site, ERF binding site and EN1 binding site 2, wherein said mutations disrupt said repressor binding site without deleting the whole repressor binding site sequence. Said method comprises the step of introducing the composition as described above into a cell such that said gene editing enzyme induces site-specific mutation(s) within said target sequence, wherein said mutations disrupt repressor binding site without deleting the whole repressor binding site sequence.

In a particular embodiment, said gene editing enzyme is selected from the group consisting of site-specific nuclease, base editor and prime editor, preferably CRISPR/cas gene editing enzyme as described above.

In a particular embodiment, said gene editing enzyme is a site-specific nuclease, more preferably CRISPR/Cas nuclease comprising a guide RNA and Cas protein, wherein said guide RNA in combination with Cas protein cleaves and induces indel and/or substitution mutation(s) within said target sequence comprising a utrophin repressor binding site selected from the group consisting of: Let7c binding site, miR-196b binding site, ERF binding site and EN1 binding site 2 and which disrupt said repressor binding site without deleting the whole repressor binding site sequence.

In another particular embodiment, said gene editing enzyme is a base or prime editor, preferably CRISPR base or prime editor which induces site-specific mutation(s) within said target sequence comprising a utrophin repressor binding site selected from the group consisting of: Let7c binding site, miR-196b binding site, ERF binding site and EN1 binding site 2 and which disrupt said repressor binding site without deleting the whole repressor binding site sequence.

Said method involves introducing gene editing enzyme such as Cas protein, base editor or prime editor and guide RNA (crRNA, tracrRNa, or fusion guide RNA or pegRNA) into a cell. Said gene editing enzyme, preferably guide RNA and/or Cas protein, base editor or prime editor as described above may be synthesized in situ in the cell as a result of the introduction of nucleic acid construct, preferably expression vector encoding said gene editing enzyme such as guide RNA and/or Cas protein, base editor or prime editor as described above into the cell. Alternatively, said gene editing enzyme such as guide RNA and/or Cas protein, base editor or prime editor may be produced outside the cell and then introduced thereto.

Said nucleic acid construct or expression vector can be introduced into cell by any methods known in the art and include, as non-limiting examples, stable transformation methods in which the nucleic acid construct or expression vector is integrated into the cell genome, transient transformation methods in which the nucleic acid construct or expression vector is not integrated into the genome of the cell and virus-mediated methods. For example, transient transformation methods include for example microinjection, electroporation or particle bombardment.

In some embodiments, said method is an in vitro method. The in vitro method is performed on a culture of cells such as cells collected from a patient.

### Engineered Cells

In another aspect, the present disclosure relates to an engineered cell obtainable or obtained by the method described above.

In particular, the present disclosure relates to an engineered cell, preferably a muscle cell which comprises site-specific mutation(s) within at least one target sequence comprising a utrophin repressor binding site selected from the group consisting of Let7c binding site, miR-196b binding site, EN1 binding site 2 or ERF binding site as described above which disrupt said repressor binding site without deleting the whole repressor binding site sequence.

The engineered cell of the disclosure may be used for ex vivo gene therapy purposes. In such embodiments, said gene editing enzyme such as guide RNA and Cas protein, base editor or prime editor, nucleic acid construct, expression vector or viral particle as described above are introduced into cells.

Said cells can be subsequently transplanted to the patient or subject. Transplanted cells can have an autologous, allogenic or heterologous origin. For clinical use, cell isolation will generally be carried out under Good Manufacturing Practices (GMP) conditions.

In a particular embodiment, the engineered cell is used for ex vivo gene therapy into the muscle.

Preferably, said cells are eukaryotic cells such as mammalian cells, these include, but are not limited to, humans, non-human primates such as apes; chimpanzees; monkeys, and orangutans, domesticated animals, including dogs and cats, as well as livestock such as horses, cattle, pigs, sheep, and goats, or other mammalian species including, without limitation, mice, rats, guinea pigs, rabbits, hamsters, and the like. A person skilled in the art will choose the more appropriate cells according to the patient or subject to be transplanted.

Said engineered cell may be a cell with self-renewal and pluripotency properties, such as stem cells or induced pluripotent stem cells. Stem cells are preferably mesenchymal stem cells. Mesenchymal stem cells (MSCs) are capable of differentiating into at least one of an osteoblast, a chondrocyte, an adipocyte, or a myocyte and may be isolated from any type of tissue. Generally MSCs will be isolated from bone marrow, adipose tissue, umbilical cord, or peripheral blood. Said cells may also be satellite cells (muscle stem cells) and mesangioblasts. Methods for obtaining thereof are well known to a person skilled in the art. Induced pluripotent stem cells (also known as iPS cells or iPSCs) are a type of pluripotent stem cell that can be generated directly from adult cells. Yamanaka et al. induced iPS cells by transferring the Oct¾, Sox2, Klf4 and c-Myc genes into mouse and human fibroblasts, and forcing the cells to express the genes (WO 2007/069666). Thomson et al. subsequently produced human iPS cells using Nanog and Lin28 in place of Klf4 and c-Myc (WO 2008/118820).

Said engineered cells may also be muscle cells. As used herein, the term “muscle” refers to cardiac muscle (i.e. heart) and skeletal muscle. As used herein, the term “muscle cells” refers to myocytes, myotubes, myoblasts, and/or satellite cells.

### Pharmaceutical Composition

The gene editing enzyme such as guide RNA and Cas protein, base editor or prime editor, nucleic acid construct, expression vector, viral particle or engineered cell according to the present disclosure is preferably used in the form of a pharmaceutical composition comprising a therapeutically effective amount of said product(s) as described above.

In the context of the disclosure, a therapeutically effective amount refers to a dose sufficient for reversing, alleviating or inhibiting the progress of the disorder or condition to which such term applies, or reversing, alleviating or inhibiting the progress of one or more symptoms of the disorder or condition to which such term applies.

The term “effective dose” or “effective dosage” is defined as an amount sufficient to achieve, or at least partially achieve, the desired effect.

The effective dose is determined and adjusted depending on factors such as the composition used, the route of administration, the physical characteristics of the individual under consideration such as sex, age and weight, concurrent medication, and other factors, that those skilled in the medical arts will recognize.

In the various embodiments of the present disclosure, the pharmaceutical composition comprises a pharmaceutically acceptable carrier and/or vehicle.

A “pharmaceutically acceptable carrier” refers to a vehicle that does not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

Preferably, the pharmaceutical composition contains vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or suspensions. The solution or suspension may comprise additives which are compatible with viral vectors and do not prevent viral vector particle entry into target cells. In all cases, the form must be sterile and must be fluid to the extent that easy syringe ability exists. It must be stable under the conditions of manufacture and storage and must be preserved against the contaminating action of microorganisms, such as bacteria and fungi. An example of an appropriate solution is a buffer, such as phosphate buffered saline (PBS) or Ringer lactate.

### Therapeutic Use

The composition comprising a gene editing enzyme, such as guide RNA in combination with the Cas protein, base editor or prime editor, nucleic acid construct, expression vector, viral particle or pharmaceutical composition as described above or isolated cell according to the present disclosure may be used as medicament, in particular for the treatment of dystrophinopathies.

Dystrophinopathies are a spectrum of X-linked muscle diseases caused by pathogenic variants in DMD gene, which encodes the protein dystrophin. Dystrophinopathies comprises Duchenne muscular dystrophy (DMD), Becker muscular dystrophy (BMD) and X-linked dilated cardiomyopathy (also referred as DMD-associated dilated cardiomyopathy).

DMD is the only gene in which pathogenic variants cause the dystrophinopathies. More than 5,000 pathogenic variants have been identified in persons with DMD, BMD or X-linked dilated cardiomyopathy. Disease-causing alleles are highly variable, including deletion of the entire gene, deletion or duplication of one or more exons, and small deletions, insertions, or single-base changes (see Darras BT, Miller DT, Urion DK. Dystrophinopathies. 2000 Sep 5 [Updated 2014 Nov 26]. In: Pagon RA, Adam MP, Ardinger HH, et al., editors. GeneReviews® [Internet]. Seattle (WA): University of Washington, Seattle; 1993-2017. Available from: https://www.ncbi.nlm.nih.gov/books/NBK1119/, as well as OMIM Entries for Dystrophinopathies 300376, 300377, 302045 and 310200).

The disclosure provides also a method for treating a dystrophinopathy according to the present disclosure, in particular Duchenne muscular dystrophy (DMD), Becker muscular dystrophy (BMD) or X-linked dilated cardiomyopathy comprising administering to a patient a therapeutically effective amount of the composition, pharmaceutical composition or isolated cell as described above.

By “therapeutically effective amount” refers to an amount effective, at dosages and for periods of time necessary to achieve the desired therapeutic result. The therapeutically effective amount of the product of the disclosure, pharmaceutical composition that comprises it or cells may vary according to factors such as the disease state, age, sex, and weight of the individual, and the ability of the product or pharmaceutical composition to elicit a desired response in the individual. Dosage regimens may be adjusted to provide the optimum therapeutic response. A therapeutically effective amount is also typically one in which any toxic or detrimental effect of the product or pharmaceutical composition is outweighed by the therapeutically beneficial effects.

As used herein, the term “patient” or “individual” denotes a mammal. Preferably, a patient or individual according to the disclosure is a human.

In the context of the disclosure, the term “treating” or “treatment”, as used herein, means reversing, alleviating or inhibiting the progress of the disease caused by dystrophin dysfunction or condition to which such term applies, or reversing, alleviating or inhibiting the progress of one or more symptoms of the disorder or condition to which such term applies.

The product of the present disclosure is generally administered according to known procedures, at dosages and for periods of time effective to induce a therapeutic effect in the patient.

The administration can be systemic or local. Systemic administration is preferably parenteral such as subcutaneous (SC), intramuscular (IM), intravascular such as intravenous (IV) or intraarterial; intraperitoneal (IP); intradermal (ID), interstitial or else. The administration may be for example by injection or perfusion. In some preferred embodiments, the administration is parenteral, preferably intravascular such as intravenous (IV) or intraarterial. The practice of the present disclosure will employ, unless otherwise indicated, conventional techniques, which are within the skill of the art. Such techniques are explained fully in the literature.

In a further aspect, the present disclosure also concerns the non-therapeutic use of the composition as described above for use to activate utrophin gene expression, for example for research tool.

In another aspect, the present disclosure relates to a kit for enhancing utrophin expression, said kit comprising a gene editing enzyme, such as guide RNA in combination with a Cas protein, base editor or prime editor, nucleic acid construct, expression vector or viral particle as described above or isolated cell according to the present disclosure.

The invention will now be exemplified with the following examples, which are not limitative.

### EXAMPLES

### 1. Materials and Methods

### Cell Culture

Human DMD myoblasts were maintained in Smooth Muscle Cell Growth Medium (C-23060, PromoCell) supplemented with 1% Penicillin Streptomycin (Invitrogen). Cells were maintained at 5% CO₂ at 37° C.

### sgRNA Design

The guides targeting the Let7c and ERF binding sites were chosen on the basis of their proximity to the mutation intended for editing and designed on the basis of the most active sgRNAs as computationally predicted by the online Benchling Tool described by Doench et al. 2016, Nat. Biotechnol. 34(2):184-191. All sgRNAs with a predicted activity score greater than 0.30 were next analyzed by the CRISPR Design tool and ranked according to the least possible number of potential off-target sites (Hsu et al. 2013. Nat Biotechnol. 2013 Sep;31(9):827-32).
hLetc1 (Letc1): ATGGATCTGAGGTAGAAAGG (SEQ ID NO: 5)
miR-196b: GTGCTTTCTTGGGTATGACA (SEQ ID NO: 7)
miR-150-133b-296-5p(II): TTATTTTAGAATAGGTTGGG (SEQ ID NO: 24)
hERB (ERB): TCTTCCGGAACAAAGTTGCT (SEQ ID NO: 9)
hLet7c2: CTGAGGTAGAAAGGTGGTCA (SEQ ID NO: 4)
hLet7c3: AAGATGGATCTGAGGTAGAA (SEQ ID NO: 6)
mLet7c2: CTGAGGTAGAAAGGTGATCA (SEQ ID NO: 3)
mLet7c4: AAGGTGGTTCTGAGGTAGAA (SEQ ID NO: 25)
EN1 (hEN1): GCTGACCCGGGAACGTAGTG (SEQ ID NO: 16).

NucleofectionChemically modified single guide RNA (Synthego) comprising 2′-O-Methyl at 3 first and last bases and 3′ phosphorothionate bonds between first 3 and last 2 bases were diluted following manufacturer’s instruction. Ribonucleoprotein complexes were formed with sgRNA and 30 pmol of Streptococcus pyogenes Cas9 protein (ratio 1:2). 2.5 × 10⁵ hDMD myoblast cells per condition were transfected with RNP using P5 Primary Cell 4D-Nucleofector X Kit (C2C12 program) in the presence of Alt-R® Cas9 Electroporation Enhancer (#1075916; IDT). Culture medium was replaced the following day, and cells were harvested for protein analysis 48 hours after electroporation.

### DNA Analysis

Genomic DNA was extracted with the QuickExtract™ DNA Extraction Solution (Lucigen, Middelton, WI, USA). 50 ng of genomic DNA were used to amplify the region that spans the cutting site of each gRNA using KAPA2G Fast ReadyMix (Kapa Biosystem, Wilmington, MA, USA). After Sanger sequencing (Genewiz, Takeley, UK), the percentage of insertions and deletions (InDels) was calculated using TIDE software (Brinkman et al. 2014, NAR 41(12):168).

### RNA Extraction and RT-qPCR

Total RNA was purified using RNeasy Micro kit (Qiagen, Hilden, Germany). RNA was reverse-transcribed using Transcriptor First Strand cDNA Synthesis Kit (Roche, Basel, Switzerland). qPCR was performed using Maxima Syber Green/Rox (Life Scientific, Thermo-Fisher Scientific, Waltham, MA, US). Utrophin A (forward primer 5′-ACGAATTCAGTGACATCATTAAGTCC-3′ (SEQ ID NO: 22), reverse primer 5′ ATCCATTTGGTAAAGGTTTTCTTCTG-3′, (SEQ ID NO: 23) mRNA expression levels were normalized using human GAPDH as a reference gene (NM_002046.6) and represented as fold changes (2^ΔΔCt) relative to the control.]. No reverse transcriptase (no-RT) and no template control (NTC) reactions were used as negative controls in each 40-cycle PCR run (Cq values NTC = undetermined, non-RT = undetermined).

### Protein Analyses

Muscles cells samples were homogenized on ice in RIPA buffer (R0278-50ml, Sigma-Aldrich) supplemented with protease inhibitors (P8340, Sigma-Aldrich). Following BCA quantification, 10 µg of total protein were heat-denatured for 5 minutes at 100° C. before loading onto NuPAGE 3- 8% TRIS Acetate Midi Gel (Novex, Life Technologies) and transferred to PVDF membranes (Millipore). Membranes were blocked for 1 hour with Odyssey Blocking buffer (926-41090; LI-COR; USA) and then incubated with the following primary antibodies for 2 hours at room temperature: mouse anti-utrophin (1:50, MANCHO3(84A)) and rabbit anti-gapdh (1:5000, MAB374, Sigma Aldrich). The Odyssey Imaging System and the Image Studio Lite software (LI-COR Biosciences; USA) were used to quantify target proteins relative to vinculin.

### Reporter Assay and Transfection

The utrophin 3′UTR is based on the human utrophin UTRN-001 (ENST00000367545.7) sequence. All utrophin 3′UTR reporter constructs were generated by GenScript Biotech (Leiden, Netherlands) and integrated in the pEZX-GA02 Gaussia luciferase (Gluc) and secreted alkaline phosphatase (SEAP) reporter cloning vector (ZX-104, Genecopoeia) downstream of the Gaussia luciferase reporter gene. Correct integration was controlled by enzymatic digestion and all plasmids were sequenced to verify the identity of the constructs. Following transformation in XL-10 bacteria, plasmid preparation was performed using NucleoSpin Plasmid kit (740588.50, Macherey Nagel) and following manufacturer recommendations. To study the impact of utrophin 3′UTR variants on the Gaussia Luciferase reporter gene expression, hDMD D52 myoblast were seeded in 96-well plates at 10,000 cells/well. The day after, cells were transfected using Lipofectamine™ 3000 (L3000008, ThermoFisher) as transfection agent. Briefly, 100 ng of the pEZX-GA02-3′UTR variant and the 0.2 ul of P3000 reagent were diluted in 5 ul of Optimen prior to be gently mixed with 0.3 ul of Lipofectamine 3000 diluted in 5 ul of Optimen. After 15 min of incubation at room temperature, the mixture was diluted with serum-free culture medium to a final volume of 100 ul. Experiments were done in triplicate. Forty eight hours after transfection, supernatant was collected for enzymatic dosages.

### Enzymatic Dosages

Gaussia luciferase activity was measured by using the following protocol: culture medium was collected and diluted in PBS1X using a 1:10 dilution. 50 ul of diluted supernatant were distributed in white 96-well OptiPlate. 11 ul of Coelenterazine (C3230-50UG, Sigma Aldrich) were diluted in 5.5 ml of PBS1X and automatically distributed. Luciferase light units were measured using the EnSpire Multimode Plate Reader ((Perkin Elmer, Courtaboeuf, France). The transfection efficiency was controlled by quantification of the SEAP using the Phospha-Light™ SEAP Reporter Gene Assay System (T1015, ThermoFisher) and a 1:20 dilution of the supernatant. Gaussia luciferase value were normalized by SEAP measurements. All condition has been performed in triplicate.

### Mice and Drug Treatment

All animal procedures were performed in accordance with the European guidelines for the human care and use of experimental animals, and animal experimentations were approved by the Ethical Committee for Animal Experimentation C2AE-51 of Evry under number APAFIS#29497-2020102611378971 v2 and DAP 2020-001-B. All C57BL/10ScSn-Dmdmdx/J (BL10/mdx) male mice were bred in CERFE (Experimental Functional Research Exploration Center) facility, Génopole.

Four-week-old mdx mice were administered by tail-vein injection with a total 10^{E}12 vector genomes of rAAV9-CMV-Cas9 and rAAV9-gmLet7c2. A SpCas9 (1) : gmLet7c2 (5) ratio was used. Control mdx mice receive a total 10^{E}12 vector genomes of rAAV9-CMV-Cas9 and rAAV9-gRosa26.1. All mice were then harvested at 9 weeks of age. For histological and molecular analysis of mouse tissues, specimens were collected immediately after animals were killed by cervical dislocation, snap frozen in liquid-nitrogen-cooled isopentane and stored at -80° C.

### Histological Analyses

Tibialis anterior (TA) muscle transverse cryosections (8 µm thickness) were prepared from frozen muscles, air dried, and stored at -80° C. Mouse sections were processed for Hematoxylin-Eosin staining as previously described [Guiraud et al., HMG. 2015]. Whole muscle sections were visualized on an Axioscan Z1 automated slide scanner (Zeiss, Germany), using the ZEN2.6 SlideScan software and a Plan APO 10×0.45 NA objective. The proportion of centrally nucleated fibres was determined by analysing the H&E images of the whole muscle section. Areas of necrosis were quantified based on the DMD_M.1.2.007 MDC1A_M.1.2.004 TREAT-NMD SOPS and performed with the Fiji ImageJ 1.49i software on the TA sections.

### Immunofluorescence

Frozen transverse muscle sections were fixed 10 min in acetone, then blocked in M.O.M.® (Mouse on Mouse) (BMK-2202, Vector Laboratories) for 30 min and incubated with the mouse monoclonal anti-utrophin (1:50, SC-33700) primary antibody overnight at 4° C. Sections were next washed in PBS and incubated with suitable Alexa Fluor secondary antibodies for 1 h at room temperature. Sections were examined under an Axioplan 2 Microscope System (Carl Zeiss, Germany).

### Statistics

Results were analysed using Prism (GraphPad Software, Inc.) and the Student’s t test with a two tailed distribution assuming equal or unequal sample variance depending of the equality of the variance (F-test). Data are presented as mean ± SEM (standard error of mean), with n indicating the number of independent biological replicates used in each group for comparison. Differences were considered significant at (*) p<0.05; (**) p<0.01 and (***) p<0.001.

### 2. Results

Using single guide RNA, the inventors target specific repressor domains on the utrophin promoter and the 3′-UTR of utrophin gene (FIG. 1). Using 3′-UTR sequence (SEQ ID NO: 43) as reference sequence, AU-Rich elements are from positions 314 to 336; miR-296-5p (I) is from positions 314-336; miR-206 is from positions 394-415; miR-150 is from positions 1508-1527; Let7c is from positions 1593 to 1616; miR- 196b is from positions 1697-1715.

In human DMD myoblasts, the inventors nucleofected a ribonucleoprotein (RNP) Cas9 and different single guides targeting the Let7c, miR-196b, miR-150/133b/296-5p(II) binding sites on the 3′UTR of utrophin and ERF binding site in the 5′UTR of utrophin gene.

Negative control corresponds to human DMD myoblasts nucleofected with Cas9 without single guide RNA. After 48 hours of treatment, the inventors observed 70% and 87 % efficacy of edition (Table 3) with single guides targeting the miR-196b and Let7c respectively associated with a significant 1.8 and 4.1-fold increase of utrophin mRNA level (FIG. 2). The inventors also used the RNP Cas9 system to disrupt the Ets-2 repressor factor binding site (ERB) in the promoter region and observed a 90% efficacy of edition (Table 3) associated with a 4.7-fold increase of utrophin mRNA level (FIG. 2).

TABLE 3

Efficacy of edition. InDel are determined by using TIDE software [Brinkman et al. 2014, NAR 41(12):168]

| | Indel % |
|---|---|
| Negative control | 0 |
| | 0 |
| Cas9 prot + gLet7c1 | 87 |
| Cas9 prot + gmiR-196b | 70 |
| Cas9 prot + gmiR-150-133b-296-5p(II) | 16 |
| Cas9 prot + gERB | 90 |

In dystrophic myoblasts, these results are superior to the ones obtained with previous published utrophin-based strategies as mentioned above. In contrast, 16 % efficacy of edition (Table 3) with no significant increase of utrophin mRNA level was observed with single guide targeting the gmiR-150-133b-296-5p(II) (FIG. 2).

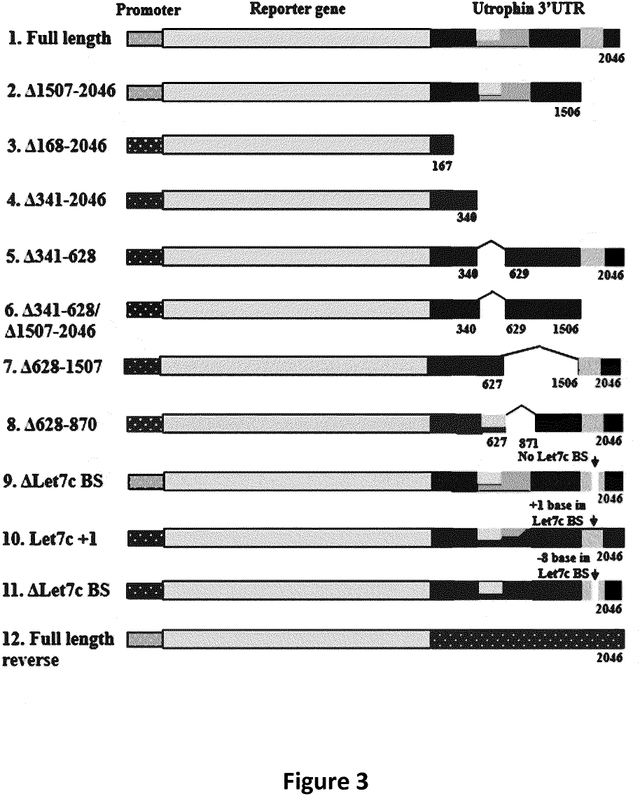

The inventors then looked for the 3′UTR responsible for downregulating UTRN expression. Therefore, several variants of the 3′UTR were designed (FIG. 3). These different 3′UTR were integrated in the pEZX-GA02, a dual reporter system. All constructs were next transfected in hDMD-D52 myoblasts. Using a reporter system (Gaussia Luciferase), the consequences of each construct/deletion on the GLuc expression could be studied. This gave some idea about the best deletion to generate to increase gene expression.

The results obtained with all constructs in hDMD D52 myoblasts are presented in FIG. 4. These data allow to define the best area to delete, corresponding to positions 341-2046 of 3′UTR (Construct C4). This deletion shows no significant difference compared to the deletion of the binding site of Let7c (construct C9), indicating that Let7c is a very interesting and probably the best sequence to target. Using the guide hLet7c1, main genetic modifications are the addition of one nucleotide and the deletion of 8 nucleotides. The disruption of the let7c binding site by the addition of one nucleotide or the deletion of 8 nucleotides are equally efficient than the complete deletion of the binding site of Let7c (comparison of constructs C9 versus C10 and C11). These data demonstrate that a single guide generating a point mutation is as efficient than a deletion generated by 2 sgRNA.

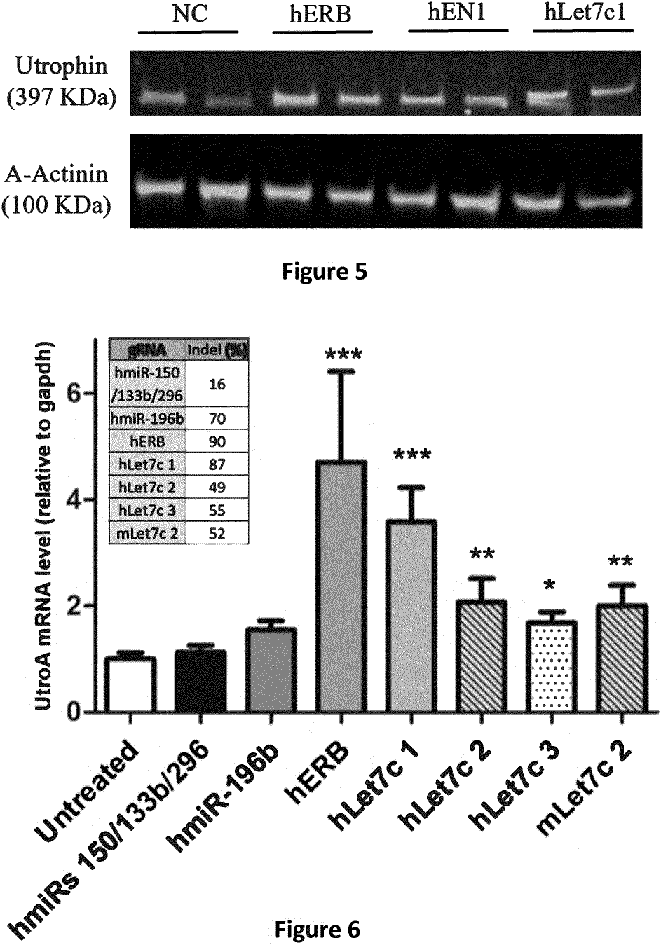

The protein results obtained with hLet7c1, hEN1 and hERB in hDMD D52 are shown in FIG. 5. After 5 days of treatment, all guide/treatment conduct to a ⅔-fold increase of utrophin A expression compared to negative control.

According to these data, targeting the Let7c binding site with one guide was the best option on the 3′UTR of utrophin. Several guides target hLet7c1 in human DMD myoblasts. The most efficient guide in human DMD myoblasts was hLet7c1 (FIG. 6). In usual condition (ratio cas9:guide 1:2 with enhancer 1×), this guide cut with a 87% efficiency and utrophin mRNA expression is increased up to 4.1-fold. Therefore, the inventors focus on Let7c and looked at the other potential guide (Let7c2 and 3). Of importance, hLet7c1 is specific to the human UTRN and the Let7c2 guide is “compatible” for the human and mouse 3′UTR sequence (one nucleotide mismatch between hLet7c2 and mLet7c2). With usual condition, the hLet7c2 and mLet7c2 cut with a 49-52% efficiency and increase utrophin mRNA expression by 2-fold (FIG. 6). The h and mLet7c2 behave in similar way and their indel profile is similar.

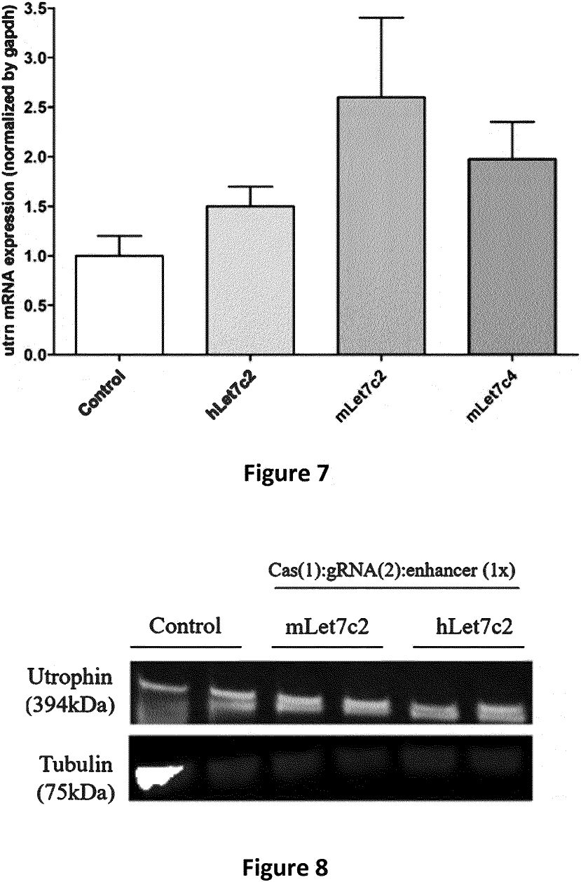

In order to test UTRN upregulation by let7c BS disruption in a DMD mouse model (mdx mice), the inventors designed two additional guides specific for mLet7c BS, mLet7c2 and mLet7c4. In C2C12 immortalized mouse myoblast cell line, mLet7c2 was the most potent showing 2-fold increase of utrophin protein expression (FIGS. 7 and 8).

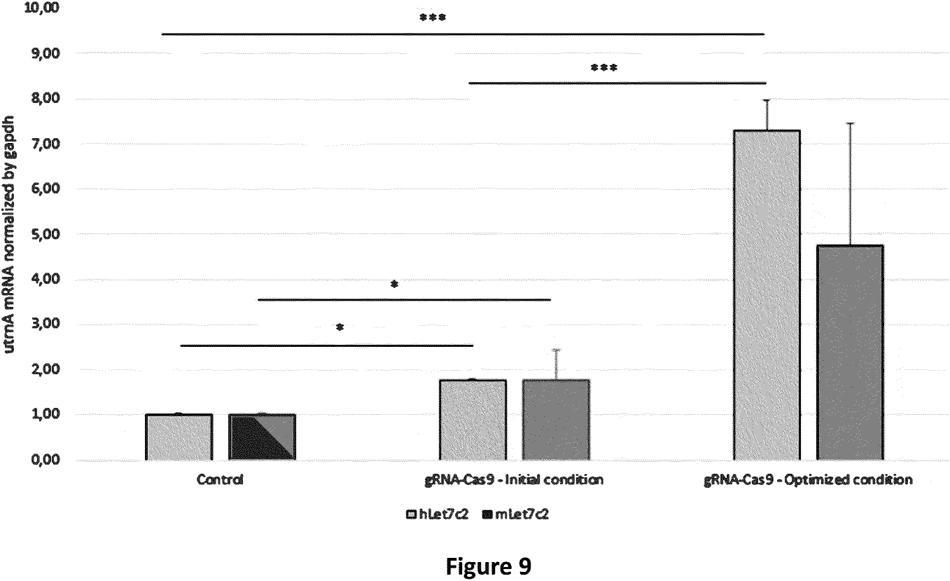

The conditions of cell treatment were modified to improve the cutting efficiency and the subsequent utrophin levels. The ratio Cas9:guide was changed from 1:2, to 1:5 and the concentration of enhancer used was increased from 1× to 5×. With these optimized conditions, the efficacy of cutting was increased up to 95% and reach a 7-fold increase of utrophin mRNA (FIG. 9). These studies were done in hDMD D52 myoblasts.

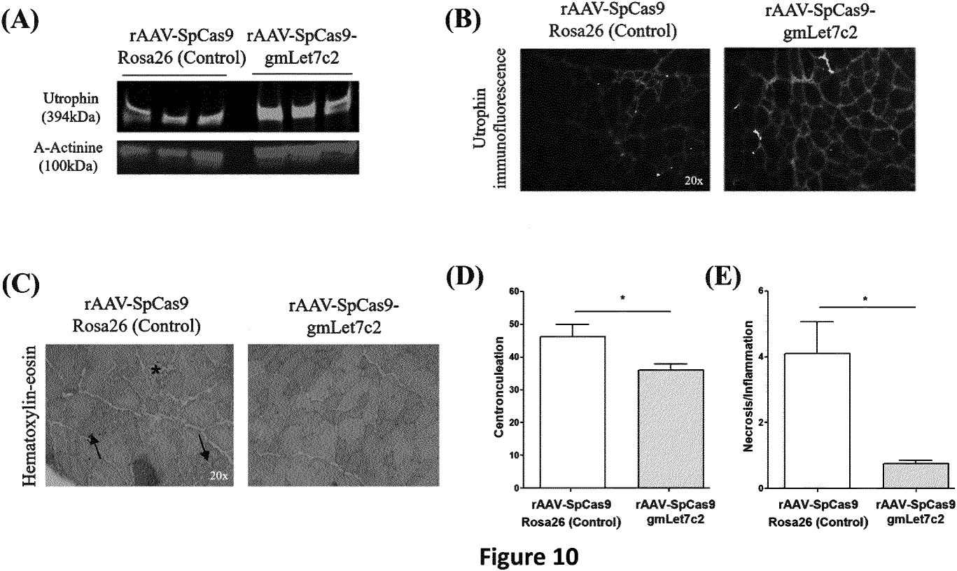

Recombinant AAV expressing Cas 9 and recombinant AAV expressing mLet7c2 were administered intravenously to mdx mice (1^{E}12vg total dose using rAAV-SpCas9/AAV-mLet7c2 ratio of 1: 5). Treatment with rAAV-Rosa26/rAAV-SpCas9 was used as control. Western blot analysis of TA muscle tissue showed a 1.6-fold increase of utrophin protein expression after 5 weeks of treatment with a 1^{E}12vg total dose of rAAV-mLet7c2/rAAV-SpCas9 compared with control (FIG. 10A). Immunofluoresence staining of muscle section confirms that utrophin signal is increased after rAAV-mLet7c2/rAAV-SpCas9 treatment and localised to the muscle membrane compared to control (FIG. 10B). Muscle from mice treated with rAAV-mLet7c2/rAAV-SpCas9 showed a significant 22% (p=0.03) decrease in centrally nucleated fibers compared to the control group in TA muscles. The necrotic muscle area in TA of mice treated with rAAV-mLet7c2/rAAV-SpCas9 significantly decreased by 82% (P = 0.03) compared to the control group (FIG. 10C). This shows that treatment of mdx mice with rAAV expressing Cas9 and rAAV expressing single gRNA targeting Let7c binding improved muscle architecture and histology mice compared to control. These results open new perspectives for the treatment of dystrophinopathies.

1-18. (canceled)

19. A method for enhancing utrophin expression in a cell, comprising introducing into a cell a composition comprising at least one gene editing enzyme capable of inducing site-specific mutation(s) within a target sequence comprising at least one repressor binding site of utrophin gene selected from the group consisting of: Ets-2-repressor factor (ERF) binding sites, homeobox protein engrailed-1 (EN1) binding site 2, Let7c binding site and miR-196b binding site, wherein said mutation(s) disrupt the repressor binding site without deleting the whole repressor binding site sequence.

20. The method of claim 19, wherein said repressor binding site is selected from the group consisting of ERF binding site consisting of sequence CGGAA, EN1 binding site 2 consisting of sequence GTAGTGG, Let7c binding site consisting of SEQ ID NO: 1, and miR-196b binding site consisting of sequence SEQ ID NO: 2.

21. The method of claim 19, wherein said repressor binding site is Let7c binding site.

22. The method of claim 19, wherein said gene editing enzyme is selected from the group consisting of a site-specific nuclease, base editor and prime editor.

23. The method of claim 22, wherein said gene editing enzyme is a CRISPR/Cas gene editing enzyme comprising a guide RNA that comprises a complementary sequence to said target sequence comprising a utrophin repressor binding site.

24. The method according to claim 22, wherein said gene editing enzyme is a CRISPR/Cas gene editing enzyme comprising a guide RNA selected from the group consisting of SEQ ID NO: 3 to 17 and 25.

25. The method according to claim 19, wherein said composition comprises at least two gene editing enzymes which are sequence-specific nucleases and wherein said nucleases are used successively in such a way that a first sequence-specific nuclease induces a first site-specific mutation event within a target sequence and once first mutation event is repaired, a second sequence-specific nuclease is used to induce a second site-specific mutation event within a target sequence.

26. A composition for enhancing utrophin expression comprising at least one gene editing enzyme capable of inducing site-specific mutation within a target sequence comprising a repressor binding site of utrophin gene selected from the group consisting of: Ets-2-repressor factor (ERF) binding sites consisting of sequence CGGAA, homeobox protein engrailed-1 (EN1) binding site 2 consisting of sequence GTAGTGG, Let7c binding site consisting of SEQ ID NO: 1 and miR-196b binding site consisting of SEQ ID NO: 2 and wherein the mutation(s) disrupt the repressor binding site without deleting the whole repressor binding site sequences.

27. The composition of claim 26, wherein the repressor binding site of utrophin gene is Let7c binding site consisting of SEQ ID NO: 1.

28. The composition of claim 27 wherein said gene editing enzyme is CRISPR/Cas gene editing enzyme comprising a guide RNA that comprises a complementary sequence to said target sequence comprising a utrophin repressor binding site.

29. The composition of claim 27, wherein said gene editing enzyme is a CRISPR/Cas gene editing enzyme comprising guide RNA comprising a sequence selected from the group consisting of SEQ ID NO: 3 to 17 and 25.

30. The composition according to claim 26, wherein said gene editing enzyme is encoded by a nucleic acid construct.

31. The composition according to claim 30, wherein said nucleic acid construct is included in a viral vector.

32. The composition according to claim 30, wherein said nucleic acid construct is included in an AAV vector.

33. The composition according to claim 26 which is a pharmaceutical composition, further comprising a pharmaceutical excipient.

34. The composition according to claim 26 which is a pharmaceutical composition for treating dystrophinopathies.

35. The composition according to claim 26 which is a pharmaceutical composition for treating a dystrophinopathy selected from the group consisting of: Duchenne Muscular Dystrophy, Becker Muscular Dystrophy and X-linked dilated cardiomyopathy.

36. An engineered cell comprising site-specific mutation(s) within at least one target sequence comprising repressor binding site of utrophin gene selected from the group consisting of: Ets-2-repressor factor (ERF) binding sites, homeobox protein engrailed-1 (EN1) binding site 2, Let7c binding site and miR-196b binding site, wherein the mutations disrupt repressor binding site without deleting the whole repressor binding site sequence.

37. The engineered cell of claim 36, wherein said Ets-2-repressor factor (ERF) binding sites consists of sequence CGGAA, homeobox protein engrailed-1 (EN1) binding site 2 consists of sequence GTAGTGG, Let7c binding site consists of SEQ ID NO: 1 and miR-196b consists of SEQ ID NO: 2.

38. The engineered cell of claim 36, wherein said repressor binding site of utrophin gene is Let7c binding site.
